(19) United States
(12) Patent Application Publication
(10) Pub. No.: US 2026/0351335 A1
(43) Pub. Date: Nov. 5, 2026

(54) ENGINEERING OF ANTIGEN-BINDING PROTEINS

(71) Applicant: Alchemab Therapeutics Ltd, London, Greater London (GB)

(72) Inventors: Jinwoo Leem, London, Greater London (GB); Jacob Galson, London, Greater London (GB); Justin Barton, London, Greater London (GB)

(73) Assignee: Alchemab Therapeutics Ltd, London, Greater London (GB)

(21) Appl. No.: 19/120,707

(22) PCT Filed: Oct. 12, 2023
(86) PCT No.: PCT/EP2023/078395

§ 371 Date: Apr. 11, 2025

(30) Foreign Application Priority Data
Oct. 12, 2023 (GB) 2215218.5

(51) Int. Cl. G16B 15/30; G06N 3/0455; G16B 30/00; G16B 40/20

(52) U.S. Cl. CPC G16B 15/30; G06N 3/0455; G16B 30/00; G16B 40/20

(57) ABSTRACT

Methods for determining whether a pair of protein chains comprising a first chain and a second chain are likely to form a functional antigen-binding protein are described The methods comprise: providing a query pair of sequences comprising the sequence of the first protein chain and the sequence of the second protein chain as input to a deep learning model configured to take as input a pair of protein chain sequences and to produce as output a score indicative of the probability that the pair of protein chains will form a functional antigen-binding protein, wherein the deep learning model comprises an encoder module and a classifier module, and wherein the deep learning module has been trained using paired training sequences from known antigen-binding proteins. The methods find use in the context of identifying antibodies from unpaired or single chain sequences. Related methods and products are described.

### FIELD OF THE INVENTION

The present invention relates to methods for engineering antigen-binding proteins such as B cell receptors, antibodies and T cell receptors by determining whether a candidate variable chain pairing is likely to be functional, such as determining whether a candidate heavy-light chain pair or a candidate α-β or γ-δ chain pair is likely to be functional, or by identifying a candidate variable chain (e.g. heavy/light, α/β, γ/δ chain) that is likely to form a functional pairing with an input chain (e.g. light/heavy, β/α, δ/γ chain). The present invention also relates to methods of providing an antigen-binding protein, such as a therapeutic antibody, derived from an input variable chain, for example a B cell receptor/antibody heavy or light chain.

### BACKGROUND TO THE INVENTION

Effective humoral immunity requires a great diversity of B cells capable of binding different antigens through their B cell receptor (BCR). The theoretical total size of the BCR repertoire in humans is estimated to be up to ~10¹⁵ variants, of which ~10⁹ are in circulation in a single individual at any time [Rees, 2020]. BCRs are comprised of two pairs of two protein chains: two heavy chains and two light chains. Each B cell expresses a (likely unique) pair of heavy and light chains to form its BCR, which is expressed on its surface, or secreted as an antibody. Over 600 million different human heavy chain sequences and approximately 70 million light chain sequences are currently catalogued in the Observed Antibody Space [Kovaltsuk et al., 2018]. Characterising the ensemble of BCRs of an individual (also referred to as an individual's BCR repertoire), has proven to be a valuable tool for understanding the biology of various diseases [Vander Heiden et al., 2017; Bashford-Rogers et al, 2019; Nielsen et al., 2020; Simonich et al., 2019] and discovering novel therapeutic antibody drugs [Krawczyk et al., 2019; Galson et al., 2020].

There are two main approaches to characterise the BCR repertoire of an individual: single B cell sequencing, and sequencing of bulk B cell populations. Single-cell sequencing is more commonly employed for antibody discovery applications, as it preserves the pairing information between the heavy and the light chains. However, single-cell sequencing has a limited throughput, and different platforms and protocols vary in their coverage of the BCR repertoire present within a single sample. Even the most advanced microfluidic systems can typically only recover the sequences for ~10⁴ B cells per sample [King et al., 2021; Eccles et al., 2020; Setliff et al., 2019]. Humans typically have ~10⁶ B cells per millilitre of blood [Mora and Walczak, 2019], meaning that single-cell approaches are not capable of characterising the full B cell diversity of even small samples. Additionally, single-cell sequencing has very specific sample requirements (for example, the cells typically have to remain viable until processed, thus requiring fresh samples processed on the day of collection, or frozen according to a specific protocol), very high costs per sample compared to bulk sequencing (single-cell sequencing being at least an order of magnitude more expensive than bulk sequencing) and requires dedicated laboratory equipment.

Sequencing of bulk B cell populations can more readily recover ~10⁷ B cell sequences per sample [Briney et al., 2019], which is significantly closer to the expected diversity in an individual. However, as B cells are lysed during library preparation, heavy-light chain pairing information is not preserved. Typically, these bulk BCR sequencing approaches focus only on the heavy chain, as it plays the dominant role in antigen binding and is much more diverse than the light chain repertoire [Kovaltsuk et al., 2018]. However, for antibody discovery, it is necessary to have both the heavy and light chain of an antibody so that it can be synthesised and functionally characterised. The gap in light chain pairing information has prompted the development of computational pairing methods [Reddy et al., 2010, Zhu et al., 2013, Raybould et al., 2021, Rakocevic et al., 2021]. However, these are limited to specific datasets and a few particular sequences within these datasets.

Similarly, cellular immunity requires a great diversity of T cells capable of binding different antigens through their T cell receptor (TCR). The total size of the TCR repertoire in humans is estimated to comprise up to ~10¹⁵ unique αβ T cell receptor (TCR) pairs [Carter et al., 2019]. While experimental approaches for paired αβ TCR sequencing have been developed (including single cell approaches [Zheng et al., 2017] and multi-cell deconvolution based approaches [Howie et al., 2015]), these remain specialised and limited in throughput. Thus, the majority of the TCR repertoire knowledge available is based on bulk-sequencing on single chain repertoires, mostly the β chain repertoire. This is inherently limited especially as it has been shown that both the α and β TCR chains are involved in alloreactivity and antigen specificity [Carter et al., 2019].

Thus, there is still a need for improved methods for identifying chain pairs such as BCR heavy-light chain pairs or TCR αβ chain pairs, from data that does not contain this pairing information.

### SUMMARY OF THE INVENTION

The problem of identifying BCR heavy-light chain pairs is far from trivial. Indeed, the diversity of the BCR repertoire results in a large search space. Additionally, while several heavy-light chain combinations can yield stable BCRs (an observation that has led some to speculate that pairing could be random [Glanville et al., 2009; Jayaram et al., 2012; DeKosky et al., 2016]), only a limited number of pairings produce functional BCRs that are capable of binding their target antigen [Teplyakov et al., 2016; Ling et al., 2018]. This indicates that functional pairing is non-random but that the determinants of functional pairings are obscured by the number of pairings that may be stable despite being non-functional. In practice, this means that finding the correct light chain for a particular heavy chain is challenging even if stable pairs could be predicted, as these would produce a significant number of solutions that require experimental validation and that would be expected to poorly validate if selected primarily based on stability.

Multiple different computational approaches have been suggested, each of which have several significant drawbacks. A first approach was based on matching the relative frequencies of BCR heavy and light chains when sequenced independently [Reddy et al., 2018]. In this study, mice were first immunised to generate a strong immune response, and then the top 4-5 most frequent heavy and light chains were chosen to pair. Beyond these top 4-5 sequences, pairing based on relative frequency was not possible. More recently, Rakocevic et al. showed that the approach only worked when the sample was dominated by a small number of high frequency B cells. Zhu et al. proposed a method termed phylogenetic pairing, which involves comparing architectures of phylogenetic trees generated from heavy and light chain sequence data. This method is limited to the examination of specific clonal expansions; in this case, known antiviral antibody lineages, rather than the entire BCR repertoire. Raybould et al. proposed an approach based on pairing structural models of heavy and light chains in silico. The approach is inherently limited by the restricted and heavily skewed availability of high-quality structural templates, and can at most identify features related to stability which do not necessarily translate to functionality. Further, the approach was only able to pair families of similar sequences rather than specific sequences (which would limit its practical applicability-which has not been validated experimentally). Thus, the present inventors have identified that current methods for computational heavy-light chain pairing are limited in that they only apply to specific datasets and sequences within these datasets. Indeed, any validated approach that exists are only applicable to datasets where both heavy and light chain sequences are available from the sample, where the data is dominated by large clonal expansions, and only facilitate pairing of a limited number of sequences within these datasets.

The present inventors further identified that for generalised application to antibody discovery, it is desirable to be able to generate a viable light chain for any given heavy chain. It is further desirable to be able to generate this using only heavy chain information as BCR repertoire bulk sequencing efforts often focus limited resources on sequencing the heavy chain, which is believed to play a more important functional role than the light chain. In order to tackle these problems, the present inventors postulated that it would be possible to use deep learning methods inspired by recent advances in natural language processing (NLP). Specifically, they postulated that deep learning models comprising encoder-decoder architectures (such as e.g. transformers [Vaswani et al., 2017]) and derived architectures such as BERT [Devlin et al., 2018] and ROBERTa [Liu et al., 2019] (encoder only architectures) or GPT [Brown et al. 2020] and Falcon [Penedo et al. 2023] should be able to learn the features of antibodies using masked language modelling in a similar way as has been used to train such models for natural language processing. They further postulated that the resulting learned representations would carry information that could be used by a classifier model to predict whether candidate pairs are likely to form functional pairs. Transformers have shown state-of-the-art results in a wide range of NLP tasks [Vaswani et al., 2017; Devlin et al., 2018; Liu et al., 2019; Rothe et al., 2020]. Thus, the inventors devised a method using a pretrained encoder (termed ‘AntiBERTa’) or decoder (termed ‘FAbCon’) that generates a learned representation of a heavy and light chain, which is fine tuned as part of a classifier for a pairing task. On multiple blind tests of single-cell datasets with known pairings, they showed that the approach predicts true pairs with high recall and precision. The approach offers a novel solution to light chain pairing, and a route to fill the gaps of bulk heavy chain sequencing. The inventors further identified that the same approach could be used to solve the problem of TCR chain pairing.

Thus, according to a first aspect, there is provided a method of determining whether a pair of protein chains comprising a first chain and a second chain are likely to form a functional antigen-binding protein, the method comprising: providing a query pair of sequences comprising the sequence of the first protein chain and the sequence of the second protein chain as input to a deep learning model configured to take as input a pair of protein chain sequences and to produce as output a score indicative of the probability that the pair of protein chains will form a functional antigen-binding protein, wherein the deep learning model comprises an encoder module and a classifier module, and wherein the deep learning module has been trained using paired training sequences from known antigen-binding proteins.

Also described according to the present aspect is a method of identifying an antigen-binding protein comprising a first protein chain and a second protein chain, the method comprising: providing a query first protein chain, providing one or more candidate second protein chain sequences; and determining whether each pair of protein chains comprising the query first protein chain and a candidate second protein chain are likely to form a functional antigen-binding protein, using a method as described above. Thus, also provided according to the present aspect is a method of identifying an antigen-binding protein comprising a pair of chains, the method comprising: providing a query first protein chain, and identifying a second protein chain by: providing one or more candidate second protein chain sequences; and determining whether the one or more candidate second chain sequences are likely to form a functional antigen-binding protein with the query first protein chain by: providing as input to a deep learning model each of one or more query pair of sequences comprising (i) the sequence of the first protein chain and (ii) the candidate second protein chain sequence, wherein the deep learning model is configured to take as input a pair of protein chain sequences or a plurality of pairs of protein sequences and to produce as output a score indicative of the probability that each pair of protein chains will form a functional antigen-binding protein or information derived therefrom, wherein the deep learning model comprises an encoder module and a classifier module, and wherein the deep learning module has been trained using paired training sequences from known antigen-binding proteins.

The methods according to present aspect may have one or more of the following features.

A score indicative of the probability that the pair of protein chains will form a functional antigen-binding protein may be a probability that the pair of protein chains will form a functional antigen-binding protein.

Identifying a second protein chain may comprise providing a plurality of candidate second chain sequences, and obtaining a score for each of the pairs of proteins comprising the first protein chain and a respective candidate second chain. The information derived from the scores may comprise a ranking of the pairs of protein chain sequences wherein pairs of protein sequences that are more likely to form a functional antigen-binding protein are ranked higher than pairs of protein sequences that are less likely to form a functional antigen-binding protein. The ranking may be based on the scores, such as e.g. a ranking by decreasing or increasing score. Thus, also provided according to the present aspect is a method of identifying an antigen-binding protein comprising a pair of chains, the method comprising: providing a query first protein chain, and identifying a second protein chain by: providing a plurality of candidate second protein chain sequences; and determining whether the candidate second chain sequences are likely to form a functional antigen-binding protein with the query first protein chain by: providing as input to a deep learning model a plurality of query pair of sequences each comprising (i) the sequence of the first protein chain and (ii) a candidate second protein chain sequence, wherein the deep learning model is configured to take as input a plurality of pairs of protein chain sequences and to produce as output respective scores indicative of the probability that each pair of protein chains will form a functional antigen-binding protein or information derived therefrom (such as e.g. a ranking of the plurality of pairs), wherein the deep learning model comprises an encoder module and a classifier module, and wherein the deep learning module has been trained using paired training sequences from known antigen-binding proteins. Similarly, also provided according to the present aspect is a method of identifying a functional antigen-binding protein comprising a pair of chains, the method comprising: providing a plurality of query antigen-binding proteins comprising a first protein chain and a second protein chain; and determining whether the query first and second chain sequences are likely to form a functional antigen-binding protein by: providing as input to a deep learning model the plurality of query pair of sequences, wherein the deep learning model is configured to take as input a plurality of pairs of protein chain sequences and to produce as output respective scores indicative of the probability that each pair of protein chains will form a functional antigen-binding protein or information derived therefrom (such as e.g. a ranking of the plurality of pairs), wherein the deep learning model comprises an encoder module and a classifier module, and wherein the deep learning module has been trained using paired training sequences from known antigen-binding proteins. The method may be use to prioritise query chain pairs/antigen-binding proteins based on the likelihood that they will form a functional antigen-binding protein predicted using the deep learning model.

A pair of chains and/or each protein chain may be referred to as “variable chains”. The wording “known chain pairs” or “known antigen-binding proteins” refers to antigen-binding proteins/pairs of variable chain sequences from antigen-binding proteins that are known to be present in antigen-binding proteins showing a desired antigen binding function, or in antigen-binding proteins that form part of at least one subject's B cell or T cell repertoire. The latter may also be referred to as “native” chain pairs. Thus, “known protein chains/antigen-binding proteins” may be proteins/chains pairs that have been previously identified (e.g. in a sample, individual, etc. including native chain pairs/proteins) and/or proteins/chain pairs that have a desired function (e.g. verified or verifiable by in vitro or in vivo testing, e.g. binding affinity to a target, expression, stability, etc.). All sequences may be amino acid sequences. The first (query) chain sequence may be a heavy chain sequence and the second sequence may be a light chain sequence. The antigen-binding protein may be a B cell receptor or antibody, or a protein derived therefrom. Thus, the antigen-binding protein may comprise a heavy-light chain pair.

The query sequence may comprise a heavy chain sequence or a light chain sequence. The corresponding chain sequence may be a light chain sequence or a heavy chain sequence.

The antigen-binding protein may be a T cell receptor, or a protein derived therefrom. The antigen-binding protein may comprise an αβ chain pair, wherein the first chain sequence is a β chain sequence or an α chain sequence, and the corresponding chain sequence is an α chain sequence or a β chain sequence. The first chain sequence may be a β chain sequence and the corresponding sequence may be an α chain sequence. The antigen-binding protein may comprise a γδ chain pair, wherein the first chain sequence is a δ chain sequence or a γ chain sequence, and the corresponding chain sequence is a γ chain sequence or a δ chain sequence. The first chain sequence may be a δ chain sequence and the corresponding sequence may be a γ chain sequence. The antigen-binding protein may be a T cell receptor or a protein derived therefrom. Thus, the antigen-binding protein may comprise an αβ chain pair or a γδ chain pair. Thus, the query sequence may comprise a β or δ chain sequence or an α or γ chain sequence. The corresponding chain sequence may be an α or γ chain sequence or a β or δ chain sequence.

The encoder module may comprise a plurality of encoders (also referred to as “encoder models”). Each encoder may take as input a protein chain sequence. The encoder module may comprise one or two transformer-based encoder models. The encoder module may comprise one or two encoders that have been pretrained using training sequences from unpaired protein chains from known antigen-binding proteins. The encoder module may comprise one or two encoders of a sequence-to-sequence model. A sequence-to-sequence model may be a recurrent neural network or a transformer. A sequence-to-sequence model may be a sequence-to-sequence transformer-based model. The recurrent neural network may be a gated recurrent unit (GRU)-based model or a long short-term memory (LSTM) model. For example, a GRU-based model may comprise a GRU-based encoder and a GRU-based decoder. The encoder may be a 4-layer bi-directional GRU, for example with a hidden dimension of 1024. The decoder may be a 4-layer, forward-only GRU, for example with a hidden dimension of 1024. A transformer is a deep learning model that uses the mechanism of attention. The transformer-based model may be a transformer model with an architecture using self-attention and point-wise, fully connected layers for both the encoder and the decoder. The encoder and/or the decoder may be composed of a stack of identical layers, such as e.g. 6, 12, 24 or 30 layers. Each layer of the encoder may have two sublayers: a multi-head self-attention layer and a position-wise fully connected feed forward network layer. Each layer of the decoder may have three sublayers: a self-attention sublayer, a layer that performs multi-head attention over the output of the encoder stack, and a feedforward network layer. The encoder module may comprise a decoder model that has been pretrained using training sequences comprising paired and unpaired protein chains from known antigen-binding proteins. The decoder may be a decoder of a decoder only transformer-based model. The encoder module may comprise an embedding layer and a decoder layer (which together may be referred to as “decoder model”) of a decoder-only autoregressive transformer model. The decoder model may use flash attention, multi-query attention and/or positional encoding. The decoder may be a 24 layer decoder with 12 attention heads in each layer, an embedding dimension of 768 and a feed-forward dimension of 3072. The decoder may be a 28 layer decoder with 16 attention heads in each layer, an embedding dimension of 1024 and a feed-forward dimension of 4096. The decoder may be a 56 layer decoder with 32 attention heads in each layer, an embedding dimension of 2048 and a feed-forward dimension of 8192. Each such model may be usable but the smallest of these models was already found to have very good performance.

The encoder module may comprise one or two encoders that use position encoding, such as absolute position encoding or relative position encoding. In embodiments, the encoder module comprises one or two encoders that use absolute position encoding. In embodiments, the encoder module comprises one or two encoders that use relative position encoding. Relative position encoding (also referred to as relative position representation) may be implemented as described in Shaw et al. (2018). An encoder that uses relative position encoding may be an encoder that uses rotary position encoding. Rotary position encoding may be implemented as described in Su et al. (2022). Relative position encoding may improve the ability of the model to capture relationships between positions in the chains. A transformer-like model (including e.g. an encoder only model or a transformer model) with relative position encoding may use relative positional information as an additional component to the keys and to the values used in a self-attention mechanism of the transformer-like model. The encoder module may comprise a decoder that uses positional encoding, such as ALiBi positional encoding (as described Press et al., 2021). The encoder module may comprise a decoder that uses rotary position embeddings as described in Su et al. (2022). For example the Falcon (falconllm.tii.ae/) and LlaMa2 (Touvron et al., 2023) models are decider only models using rotary position embeddings.

The encoder module may comprise one or two encoder models of a sequence-to-sequence model that has been trained using masked language modelling. Alternatively, the encoder may have been trained using span-based masked language modelling (see e.g. Joshi et al. 2020) Training an encoder model may comprise training the model to replace randomly masked positions of training amino acid sequences. The model may have been trained using masked language modelling wherein 15% of positions are masked during training and/or wherein masked positions are replaced with mask tokens, a random amino acid, or the original amino acid at the position. The encoder module may comprise one or two encoders that have been pretrained using training sequences comprising unpaired first and second protein chains from known antigen-binding proteins. The training sequences used for pretraining may comprise at least 1 million, at least 2 million, at least 5 million, at least 10 million, at least 20 million or at least 50 million individual sequences. The encoder module may comprise two copies of an encoder that has been pretrained using training sequences comprising unpaired first and second protein chains from known antigen-binding proteins. The encoder module may comprise an encoder that has been pretrained using training sequences comprising the concatenation of a sequence from a first chain and a sequence from a second protein chain from known antigen-binding proteins, wherein the concatenated sequences comprise sequences from unpaired protein chains.

The decoder may be a decoder model of a generative language model, wherein the decoder model has been trained using causal language modelling (i.e. next token prediction task) or span modelling (also referred to as “span-based masked language modelling”). The encoder module may comprise a decoder that has been pretrained using training sequences comprising unpaired first and second protein chains from known antigen-binding proteins and paired first and second protein chains from known antigen-binding proteins. The training sequences may comprise at least 500 million, 600 million or 700 million individual sequences and/or at least 1, 1.5 or 2 million paired sequences. The encoder module may comprise a decoder that has been pretrained using training sequences each comprising a single chain or the concatenation of a sequence from a first chain and a sequence from a second protein chain from a known antigen-binding protein.

The deep learning model may further comprise a cross-attention module that takes as input the output of the encoder module, and produces an output used by the classifier module to provide the score indicative of the probability that the pair of protein chains will form a functional antigen-binding protein. The cross-attention module may comprise one or more cross-attention blocks, wherein each cross-attention block comprises a self-attention layer and a cross attention layer. The cross-attention module may comprise a plurality of cross-attention blocks. The cross-attention module may comprise one or more cross-attention blocks comprising a cross-attention layer and a self-attention layer, and each cross-attention layer and self-attention layer may comprise a plurality of attention heads. Each self-attention layer may comprise one or more attention heads that attend to the output of a first encoder taking as input the first chain sequence. Each cross-attention block may further comprise a residual connection between the output of a first encoder and the output of the self-attention layer. Each cross-attention layer may comprise one or more attention heads that attend to: (i) the output of the self-attention layer or the output of a first encoder taking as input the first chain sequence, and (ii) the output of a second encoder taking as input the second chain sequence. Each cross-attention block may further comprise a residual connection between the output of a first encoder and the output of the cross-attention layer. Each self-attention layer may comprise 2, 3, 4, 5, 6, 12 or more attention heads. Each cross-attention layer may comprise 2, 3, 4, 5, 6, 12 or more attention heads. Each cross-attention block may comprise a self-attention layer and a cross-attention layer. Each cross-attention block may comprise residual connections between the output of the self-attention layer and/or cross attention layer, and the output of a first encoder that takes as input the first chain sequence. A self-attention layer and/or a cross-attention layer output may be layer normalised prior to being provided as input to a subsequent layer or module. The classification module may comprise a softmax layer that produces a score between 0 and 1 that can be interpreted as the probability that a pair of input protein chains will form a functional antigen-binding protein. The classification module may comprise one or more of: a dimensionality reduction layer, a regularisation mechanism, and a layer with an activation function. A dimensionality reduction layer may comprise an attention pooling layer, or an average pooling layer. A regularisation layer may comprise a dropout mechanism. An activation function may be selected from Tanh, Leaky ReLU, GeLU, SmeLU, Swish and ReLU. The activation function in each layer may be independently selected from Swish and ReLU. The deep learning model may take as input a plurality of query pairs of chain sequences and produces as output a respective score indicative of the probability that each query pair of protein chains will form a functional antigen-binding protein and/or a ranking of the plurality of query pairs of chain sequences such that query pairs of protein sequences that are more likely to form a functional antigen-binding protein are ranked higher than query pairs of protein sequences that are less likely to form a functional antigen-binding protein. For example, the deep learning model may take as input 8, 16, 32 or 64 pairs of query sequences, such as e.g. 32 pairs. Such a deep learning model may be advantageously able to provide predictions for many candidate chain pairings with high computational efficiency.

The paired training sequences from known antigen-binding proteins may comprise paired training heavy and light chain sequences from single B cell sequencing data. The training data may comprise one or more datasets each previously obtained by single B cell sequencing of samples obtained from subjects or by sequencing of libraries derived therefrom. The training data may further comprise paired training heavy and light chain sequences from known antibodies/B cell receptors. For example, the training data may comprise paired training heavy and light chain sequences from one or more antibody/BCR databases, from one or more known therapeutic antibodies/BCRs, and/or from one or more antibodies/BCRs that are known to have a desired binding function. The training data may comprise paired training heavy and light chain sequences from naïve B cell receptor libraries. The training data may comprise paired training heavy and light chain sequences from antigen-experienced B cell receptor libraries. Thus, the training data may comprise paired training heavy and light chain sequences obtained from subjects that have been exposed to one or more specific antigens. The training first and second chain sequences from known chain pairs may comprise paired training α and β chain sequences from single T cell sequencing data. The training data may comprise one or more datasets each previously obtained by single T cell sequencing of samples obtained from subjects or by sequencing of libraries derived therefrom. The training data may further comprise paired training first and corresponding chain sequences from known T cell receptors. For example, the training data may comprise paired training α and β chain sequences from one or more T cell receptor databases, from one or more known therapeutic TCRs, and/or from one or more TCRs that are known to have a desired binding function. The training data may comprise paired training α and β (or δ and γ) chain sequences from naïve T cell receptor libraries. The training data may comprise paired training α and β (or δ and γ) chain sequences from antigen-experienced T cell receptor libraries. Thus, the training data may comprise paired training α and β (or δ and γ) chain sequences obtained from subjects that have been exposed to one or more specific antigens. The training first and second chain sequences from known chain pairs may comprise paired training chain sequences wherein each pair comprises a chain sequence that comprises or consists of: a V-gene sequence or identifier, a J-gene sequence or identifier, and a junction sequence, and optionally a D-gene sequence or identifier. The training first and corresponding chain sequences from known chain pairs may comprise paired training chain sequences wherein each pair comprises a chain sequence that comprises or consists of: a chain sequence that comprises or consists of: a V-gene sequence or identifier, a J-gene sequence or identifier, and a junction sequence. Reference to a V-gene or J-gene may refer to the amino acid sequence corresponding to the respective gene. The training data may comprise at least 80,000, at least 100,000, at least 120,000, at least 150,000, at least 500,000, or at least 1500,000 pairs of training sequences, for example training heavy and light chain sequences. Advantageously, the training data may comprise at least 1,500,000 pairs of training heavy and light chain sequences. The training data may comprise mammalian, such as e.g. human pairs of chain sequences. The training data may comprise mammalian heavy and/or light chain sequences.

The training data may comprise human heavy and/or light chain sequences. The training data may comprise training pairs of sequences from the same species as the query sequence. The training data may comprise at least 50%, at least 60%, at least 70%, at least 80%, at least 90%, or at least 95% sequences from the same species as the query sequence(s). The query sequence(s) may be sequence that are not present in the training data. A query sequence may be a sequence that has been obtained from a sample from a subject that has a desired characteristic, such as a desired phenotype. For example, the subject may have a particular clinical characteristic.

The training data may comprise simulated training sequences. The training data may comprise simulated paired sequences, paired simulated sequences or simulated unpaired training sequences. Simulated data may comprise sequences obtained using methods for simulating antigen-binding protein sequences, such as e.g. immuneSIM (Weber et al., 2020) and/or methods for simulating protein sequences such as ProGen2 (Nijkamp et al., 2022). The known protein pairs may be referred to as a “positive set” of pairs. The training data may comprise a negative set of pairs of chain sequences that are not expected to form functional antibody-binding proteins. The negative set may comprise randomly paired first and second protein chain sequences. Instead or in addition to this, the negative set may comprise simulated pairs of sequences or pairs of simulated sequences. Instead or in addition to this, the negative set may comprise pairs of sequences from antigen-binding proteins previously determined to be non-functional. Pairs may be determined to be non-functional according to one or more predetermined functionality criteria. For example, pairs that could not be expressed experimentally, or that failed to bind a target may be considered non-functional. Thus, the training data may further comprise a negative set comprising randomly paired first and second protein chain sequences. The randomly paired first and second protein chain sequences may have been obtained or may be obtained as part of the method by re-pairing the paired training sequences from known antigen-binding proteins and/or by randomly pairing unpaired training sequences. The negative set may comprise paired first and second protein chain sequences from antigen-binding proteins previously determined to be non-functional. The known paired training sequences (positive set) may be associated with a first label and the randomly paired/negative set training sequences may be associated with a second label. The training data may comprise pairs associated with non-binary labels. The training data may comprise a positive set of pairs associated with a score that can take a plurality of values (up to and including a continuous score, where the continuous score may be bounded for example between 0 and 1). For example, pairs in a positive set may be associated with a score indicative of “nativeness” or “functionality”. Such a score may for example reflect functional information associated with known pairs, such as e.g. binding affinity, or any other metric associated with binding strength. The training data may comprise a negative set of pairs associated with a single value (e.g. 0) or a plurality of values, such as e.g. values indicative of confidence in the pair being non-functional.

The training data may further comprise unpaired training first and/or second sequences. These may be used to pretrain an encoder or decoder of the encoder module. The unpaired training first and/or second chain sequences may have any of the features of sequences described in relation to the paired sequences. In particular, the unpaired chain sequences may be the same type of sequences as the paired sequences (e.g. where the paired training sequences are heavy and light chain pairs, the unpaired training first/second chain sequences may comprise unpaired heavy and/or light chains), may comprise sequences from the same organisms (e.g. may comprise mammalian and/or human sequences, may comprise sequences from one or more organisms, may comprise sequences from naïve libraries and/or antigen exposed libraries, etc.), may comprise the same information (such as e.g. gene segments identifiers, sequences and combinations thereof). The unpaired training sequences may comprise some or all of the first and/or second sequences that are present in the paired training sequences. Advantageously, the unpaired training sequences may comprise more first chain sequences and/or more second chain sequences than the paired training chain sequences. The first (e.g. query) chain sequence may comprise or consist of: a V-gene sequence or identifier, a J-gene sequence or identifier, and a junction sequence, and optionally a D-gene sequence or identifier. The second chain (e.g. corresponding) sequence(s) may comprise or consist of: a V-gene sequence or identifier, a J-gene sequence or identifier, and a junction sequence. The format of the first and second chain sequences is related to the format of training chain sequences. Thus, a deep learning model that has been trained using training chain sequences comprising or consisting of: a V-gene sequence or identifier, a J-gene sequence or identifier, and a junction sequence, and optionally a D-gene sequence or identifier, may accept as input chain sequences comprising or consisting of these components. Similarly, a deep learning model that has been trained using training chain sequences comprising or consisting of: a V-gene sequence or identifier, a J-gene sequence or identifier, and a junction sequence, may accept as input chain sequences comprising or consisting of these components. The query sequence may comprise or consist of one or more first chain CDR sequence(s). The second sequence may comprise or consist of one or more corresponding chain CDR sequence(s). The first and/or second sequences may comprise or consist of a CDR3 sequence. The first and second protein chains may be chains that have different ranges of lengths and/or different domain architectures.

Providing a pair of protein chain sequences as input to the deep learning model (whether for training or prediction) may comprise encoding each of the protein chain sequences using a predetermined encoding scheme. According to an encoding scheme, each amino acid may be individually encoded. In embodiments, sequences are encoded using tokens that each correspond to an individual k-mer. Providing a pair of sequences to the deep learning model may comprises encoding the sequences using an encoding scheme wherein each amino acid is individually encoded. For example, a different token may be provided for each possible amino acid. Providing a pair of sequences to the deep learning model may comprises encoding the sequences using an encoding scheme wherein each gene sequence identifier corresponds to an individual token. Providing a pair of sequences to the deep learning model may comprises encoding the query sequence using an encoding scheme wherein each amino acid corresponds to an individual token. Providing a pair of sequences to the deep learning model may comprises encoding the query sequence using an encoding scheme wherein a chain sequence is preceded or followed by a special token indicating whether the protein chain sequence is a first or second protein chain (e.g. a heavy-H, or light-L chain). Providing a pair of sequences to the deep learning model may comprises encoding each sequence using an encoding scheme wherein sequences (i.e. sequences that are available as full sequences rather than gene identifiers) are encoded using tokens that each correspond to an individual k-mer (such as e.g. using byte-pair encoding). Each sequence may be encoded using overlapping k-mers. The k-mer may have any length that is shorter than the expected length of the chains. For example, the k-mers may be of length between 1 and 100, between 2 and 100, between 2 and 50, between 2 and 20, or between 2 and 10. The k-mers may be of length 1 to 5. The k-mers may be of fixed length. For example, a fixed k-mer length of 1, 2, 3, 4 or 5 may be used. A k-mer of length 1 is equivalent to encoding each character (e.g. each amino acid) individually. A k-mer of length k>2 (such as e.g. 3) may be used as part of an encoding scheme that uses overlapping or non-overlapping k-mers. Overlapping k-mers may overlap by different extents. For example, k-mers of length 3 may overlap by 1 or 2 characters. In a scheme using a k=3, each token corresponds to a unique set of 3 characters (e.g. a motif of 3 amino acids). The unpaired training data may have been filtered to exclude any sequence comprising particular region that has a length outside of a respective predetermined range of lengths. For example, any sequence that has fewer than 20 amino acids before a CDR1 region, fewer than 10 amino acids after a junction region, a CDR1 regions with a length outside of the range of 5-12 amino acids, a CDR2 region with a length outside of the range of 1-10 amino acids, and/or a CDR3 region with a length outside of a range of 5-38 amino acids may be excluded from the unpaired training data. The paired training data may have been filtered to exclude any pairs comprising a junction sequence (in the first and/or corresponding chain) that is outside of a predetermined range of lengths. In other words, the training data may not comprise any pairs comprising a first (e.g. heavy) chain junction that is outside of a predetermined range of lengths and/or a second (e.g. light) chain junction that is outside of a predetermined range of lengths. For example, pairs comprising a heavy chain junction sequence below a predetermined length, such as e.g. 3, 4, 5, 6, 7, 8, 9 or 10 amino acids, may have been excluded. As another example, pairs comprising a heavy chain junction sequence above a predetermined length, such as e.g. 25, 26, 27, 28, 29, 30, 31, 32, 33, 34, or 35 amino acids, may have been excluded. As another example, pairs comprising a light chain junction sequence below a predetermined length, such as e.g. 3, 4, 5, 6, 7, 8, 9 or 10 amino acids, may have been excluded. As another example, pairs comprising a light chain junction sequence above a predetermined length, such as e.g. 15, 16, 17, 18, 19, 20, 20, 21, 22, 23, 24, 25, 26, 27, 28, 29, or 30 amino acids, may have been excluded. The predetermined length may be the same or different for the junction sequences in the corresponding (e.g light) chain and in the first (e.g. heavy) chain of a pair. In a specific example, pairs comprising a heavy chain junction sequence of fewer than 7 amino acids may have been excluded and/or pairs comprising a heavy chain junction sequence of more than 30 amino acids may have been excluded. Instead or in addition to this, pairs comprising a light chain junction sequence of fewer than 7 amino acids may have been excluded and/or pairs comprising a light chain junction sequence of more than 20 amino acids may have been excluded. The query sequence or sequence pair may comprise one or more gene sequence identifiers and the method may further comprise replacing the one or more gene sequence identifiers by the corresponding germline sequence. The deep learning model may be a transformer-based model comprising an encoder that has been pre-trained using unpaired training first and/or corresponding chain sequences and a decoder or a bidirectional encoder that has been pre-trained using unpaired training corresponding and/or first chain sequences. The encoder module may comprise a BERT model or a variant thereof, such as e.g. BERT, ROBERTa, DistilBERT, or RoFormer (Su et al., 2022). The encoder module my comprise an encoder trained using unpaired training first and second chain sequences. Alternatively, the encoder module may comprise a model trained using training first (e.g. heavy or light) chain sequences, and a model trained using second (e.g. light or heavy) chain sequences. When the encoder module comprises two encoders trained using unpaired training first and second chain sequences, the two encoders may be the same pre-trained model. Thus, the two encoders may be initialised using pre-trained models that have the same architecture with the same parameters. The unpaired training chain sequences may comprise full length sequences for the variable region of the second chain. The unpaired training chain sequences may comprise full sequences for the variable region of the first chain. Alternatively, the encoder module may comprise a decoder that has been pretrained using unpaired training sequences and paired training sequences. For example, the decoder may take as input a string comprising an encoding for a first chain or a second chain, preceded by a token indicating the type of chain, and optionally comprising one or more padding tokens. The decoder may also be able to take as input a string comprising an encoding for a first chain and an encoding for a corresponding second chain (i.e. chains of a pair, each preceded by a token indicating the type of chain, and optionally comprising one or more padding tokens. The deep learning model may have been trained using paired first and second (e.g. heavy and light) chain sequences from known chain pairs, wherein said sequences do not comprise full length sequences for the variable region of the second chain and/or the first chain. In such embodiments, the deep learning model may have been trained by obtaining paired training sequences that comprise full length sequences for the variable region of the corresponding chain and/or the first chain by imputing missing sequence information. Imputing missing sequence information may comprise replacing a gene identifier by the corresponding germline sequence. Imputing missing sequence information may comprise using the pre-trained encoder to predict a full-length sequence for each of the paired training first (e.g. heavy) and/or second (e.g. light) chain sequences from a partial sequence. Alternatively, the unpaired training second (e.g. light) chain sequences and/or the unpaired training first (e.g. heavy) chain sequences may have been converted to a format that matches the format of the respective paired training sequences prior to pre-training the encoder(s).

Providing a query first protein chain or a query first protein chain of a query pair may comprise obtaining the sequence of the query first protein chain from a user through a user interface, from a computing device, from a sequence acquisition means or a computing device associated with a sequence acquisition means, from a database or other computer readable medium. Providing a query first protein chain or a query first protein chain of a query pair may comprise sequencing a sample comprising genetic material encoding for an antigen-binding molecule comprising the query sequence. Obtaining the query sequence may comprise performing B cell bulk sequencing of a sample comprising B cells, T cell bulk sequencing of a sample comprising T cells, or bulk sequencing of a sample comprising any other cells expressing an antigen-binding molecule comprising the query sequence, or genetic material derived therefrom, such as a B cell receptor library or a T cell receptor library. Providing a query first protein chain or a query first protein chain of a query pair may comprise obtaining a sample comprising B cells, T cells or other cells expressing an antigen-binding molecule comprising the query sequence, or genetic material derived therefrom, such as a B cell receptor library or T cell receptor library. Providing one or more candidate second protein chain sequences may comprise obtaining the sequences of the candidate second protein chains from a user through a user interface, from a computing device, from a sequence acquisition means or a computing device associated with a sequence acquisition means, from a database or other computer readable medium. The one or more candidate second protein chain sequences may be known second chain protein sequences or simulated second chain protein sequences. Known second chain protein sequences may be sequences of second chains that have been previously observed (e.g. in a sample, individual, etc.) or that are known to have a predetermined function (e.g. antigen binding proteins previously shown to bind a particular target, to be expressed in a sample, etc.). Providing a query sequence (or pair of sequences) may comprise obtaining the query sequence (or sequence pair) from a user through a user interface, from a computing device, from a sequence acquisition means or a computing device associated with a sequence acquisition means, from a database or other computer readable medium. Providing a query sequence or sequence pair may comprise sequencing a sample comprising genetic material encoding for an antigen-binding molecule comprising the query sequence. Providing a query sequence may comprise obtaining a sample comprising B cells, T cells or other cells expressing an antigen-binding molecule comprising the query sequence, or genetic material derived therefrom, such as a B cell receptor library or T cell receptor library. Providing a query sequence may comprise sequencing a sample comprising genetic material encoding for an antigen-binding molecule comprising the query sequence, for example by performing B cell bulk sequencing of a sample comprising B cells (or any other cells expressing an antigen-binding molecule comprising the query sequence, or genetic material derived therefrom, such as a B cell receptor library). Providing a query sequence may comprise obtaining a sample comprising B cells, or other cells expressing an antigen-binding molecule comprising the query sequence, or genetic material derived therefrom, such as a B cell receptor library.

The methods may comprise determining a probability for a plurality of pairs and ranking the plurality of pairs using the determined probabilities. The methods may further comprise providing one or more identified second protein chains/pairs, a part thereof or information derived therefrom, and/or one or more probabilities that one or more query pairs form a functional antigen-binding protein or information derived therefrom (such as e.g. a ranking of query pairs according to their score/probability of forming a functional antigen-binding pair) to a user through a user interface. The methods may further comprise predicting a score indicative of the probability that each one or more query pairs comprising respective candidate second protein chain sequences form a functional antigen-binding protein and identifying a candidate second protein chain sequence by applying one or more criteria on the score/probability. The one or more criteria may be individually selected from: the score/probability being above a predetermined cutoff, the score/probability being the highest predicted score/probability of a set of candidate second protein chain sequences, and the score/probability being in a predetermined top percentile of the predicted probability of a set of candidate second protein chain sequences.

According to a second aspect, there is provided a method of providing antigen-binding protein chain pairings for a plurality of query sequences comprising a first chain sequence, the method comprising: performing the method of any embodiment of the first aspect for each of the query sequences. The plurality of query sequences may be heavy or light chain sequences obtained by bulk B cell repertoire sequencing. The plurality of query sequences may comprise at least 10, at least 100, at least 1000, at least 10,000, or at least 100,000 sequences. The plurality of query sequences may have been obtained by bulk B cell sequencing of the heavy or light chain repertoire in a sample, such as a sample from a subject. The plurality of sequences may be a subset of a set of sequences obtained by bulk B cell sequencing of the heavy or light chain repertoire in a sample. The method according to the present aspect may have any of the features described in relation to the first aspect.

According to a third aspect, there is provided a method of providing an antigen-binding protein having a desired property, the method comprising: providing one or more query sequences comprising a first chain sequence, wherein at least one of the one or more query sequences is likely to have the desired property, and identifying a corresponding chain sequence for each of the one or more query sequences using the method of any embodiment of the first aspect. The method may have any one or more of the following features.

The method may further comprise obtaining one or more candidate antigen-binding proteins each comprising one of the query sequences and one or more identified second sequences. The method may further comprise testing the one or more candidate antigen-binding proteins for the desired property. The method of the present aspect may have any of the features described in relation to the first or second aspects. The one or more candidate antigen-binding proteins may be antibodies or fragment thereof. Sequences derived from an identified chain pairing may include sequences: that comprise the same CDRs but with different framework regions, sequences that contain one or more mutations compared to the identified chain pairing, and sequences that contain one or more fragments of the identified chain pairing. Obtaining a candidate antigen-binding protein may comprise identifying a coding sequence for the candidate antigen-binding protein and expressing the sequence in a suitable expression system (such as e.g. in a suitable host cell). The desired property may be a desired binding property (such as e.g. the ability to bind one or more targets, the ability to bind one or more targets with an affinity above one or more respective thresholds, etc.), a desired expression property (such as e.g. an increased expression level compared to a standard in one or more expression systems, an expression level above a predetermined level in one or more expression systems, a yield above a predetermined level in one or more expression systems, etc.), a desired stability property (such as e.g. a stability above a certain threshold in one or more conditions), or a combination thereof. The desired property may include the ability to bind a predetermined target. Testing the one or more candidate antigen-binding proteins for the desired property may comprise identifying one or more antigens that the one or more candidate antigen-binding proteins bind(s) to, for example by testing for binding to one or more candidate antigens. Testing the one or more candidate antigen-binding proteins for the desired property may comprise identifying one or more antigens that the one or more candidate antigen-binding proteins is/are likely to bind to, for example by comparison with one or more antibodies with known targets. The antigen-binding protein may be a therapeutic antibody, and the desired property may comprise binding of a therapeutic target. An antigen-binding protein may also be referred to herein as “immune protein”.

Testing the one or more candidate antigen-binding proteins for the desired property may comprise identifying the presence or absence of a desired phenotype in an organism (such as e.g. an animal model) or cell expressing the one or more candidate antigen-binding proteins. Identifying the presence of a desired phenotype may comprise expressing the one or more candidate antigen binding proteins in one or more model cells (e.g. one or more cell lines) or organisms (such as e.g. one or more animal models). The method may further comprise optimising the sequence of at least one of the one or more candidate antigen-binding proteins. Optimising the sequence of a candidate antigen-binding protein may be performed for example using any antibody optimisation technique known in the art. Optimising the sequence of a candidate antigen-binding protein may be performed using information from the sequence data from which the chain pairing was identified, for example by analysing sequences similar to the input sequence from which the chain pairing was identified. Methods for optimising antigen-binding proteins are known in the art and include the methods described in Mason et al. [2021], Seeliger et al., [2015], Warszawski et al. [2019], Hsiao et al. and Richardson et al. [2021], amongst others. Any of these methods could be used within the context of the present invention.

The query sequence may comprise the heavy chain sequence (or part of the heavy chain sequence) of a known antibody. Thus, the first chain may be a heavy chain sequence or a part of a heavy chain sequence of a known antibody. The query sequence may have been obtained by bulk BCR sequencing of the heavy chain repertoire in one or more samples. The method may comprise the step of obtaining the query sequence by bulk BCR sequencing of the heavy chain repertoire in one or more samples. The one or more samples may be from one or more subjects. The one or more subjects may have been identified as having a desired characteristic, such as e.g. a particular clinical phenotype or clinically relevant characteristic such as a biomarker profile. For example, the one or more subjects may be resilient to a particular disease or condition. The disease or condition may be selected from a cancer (such as e.g. breast cancer), a neurodegenerative disease (such as e.g. amyotrophic lateral sclerosis), and an infectious disease (such as e.g. COVID-19). The method may comprise identifying a chain pairing (e.g. a heavy-light pairing) for a plurality of query chain sequences (e.g. heavy chain sequences) selected from the first (e.g. heavy) chain sequences identified in the one or more samples, thereby obtaining a set of chain pairings (e.g. heavy-light chain pairings). The method may further comprise identifying one or more targets by screening antibodies from the same source(s) as the one or more samples against a plurality of candidate peptides. The plurality of candidate peptides may be selected based on the species from which the one or more samples originate. For example, the source of the one or more samples may be one or more human subjects and the antibody repertoire(s) from the same source(s) as the one or more samples may be screened against a set of candidate peptides representative of the human peptidome to select a plurality of candidate peptides. Identifying an antigen that the one or more candidate antigen-binding proteins bind(s) to may comprise using one or more targets identified by screening antibodies from the same source(s) as the one or more samples against a plurality of candidate peptides. The method may further comprise filtering the set of identified chain pairings based on one or more criteria. The one or more criteria may apply to the identity of an antigen or set of antigens that a candidate antigen-binding protein bind(s) to or is predicted to bind to. Providing one or more query sequences may comprise providing a first query (e.g. heavy) chain sequence and a second query (e.g. heavy) chain sequence, and identifying a second (e.g. light) chain sequence for each of the one or more query sequences may comprise identifying one or more second (e.g. light) chain sequence(s) for the first query sequence and one or more second (e.g. light) chain sequence(s) for the second query sequence. The method may further comprise comparing the former second chain sequence(s) and the latter corresponding chain sequence(s) to identify one or more light chains that may be suitable for use as the common second (e.g. light) chain of a bispecific antibody that includes both of the first (e.g. heavy) chains. For example, the one or more candidate second chain sequences may be the same or at least partially overlapping for the first query and second query, and the one or more candidate second chain sequences that satisfy one or more criteria applied to the predicted probabilities that the candidates will form a functional pair with the first and second query may be identified as suitable for use as the common second chain of the bispecific antibody. According to a fourth aspect, there is provided a method of providing a tool for predicting whether a pair of protein chains is likely to form a function antigen-binding protein or for identifying an antigen-binding protein comprising a pair of chains, the method comprising: providing training data comprising training first and second/corresponding protein chain sequences from known antigen-binding proteins, and training a deep learning model to take as input one or more pair of protein chain sequences and to produce a score indicative of the probability that the/each pair of protein chains is/are part of a functional antigen-binding protein (or information derived therefrom), using the training data. The deep learning model comprises an encoder module and a classifier module. The method of the present aspect may have any of the features described in relation to the first aspect.

The method may have any one or more of the following features. Providing training data may comprise providing unpaired training first and second chain sequences. The unpaired training first and second chain sequences may be referred to as pre-training data. The encoder module may comprise one or more encoders or decoders. The method may further comprise training a sequence-to-sequence model using unpaired training first and/or second (e.g. heavy and/or light) chain sequences, and using the encoder of the pre-trained model to initialise the encoder(s) of the encoder module. The encoder module may comprise one or two encoders that may each be encoders a BERT model or a variant thereof, such as e.g. BERT, ROBERTa, RoFormer, SpanBERT (Joshi et al. 2020), or DistilBERT, or one or two decoders that may each be decoders of a decoder only language model such as Falcon, LlaMa, GPT3 and variants thereof. The first and second transformer based models may each comprise a ROBERTa model, a BERT model or a RoFormer model. The encoder module may comprise a decoder only model such as a Falcon model (e.g. an embedding layer and decoder layer of a Falcon model). The method may further comprise providing the trained deep learning model to a user. The methods described herein are computer implemented unless context indicates otherwise, such as e.g. where a sample is obtained, processed, analysed, or a molecule or composition produced, tested or used for any other purpose.

According to a fifth aspect, there is provided a system comprising: a processor; and a computer readable medium comprising instructions that, when executed by the processor, cause the processor to perform the steps of the method of any embodiment of any preceding aspect. The instructions may case the processor to perform the steps of the method of any embodiment of the first to fourth aspects.

According to a sixth aspect, there is provided one or more computer readable media comprising instructions that, when executed by one or more processors, cause the one or more processors to perform the steps of the method of any embodiment of any preceding method aspect. The instructions may case the processor to perform the steps of the method of any embodiment of the first to fourth aspects.

According to a seventh aspect, there is provided a computer program product comprising instructions that, when executed by one or more processors, cause the one or more processors to perform the steps of the method of any embodiment of any preceding method aspect. The instructions may case the processor to perform the steps of the method of any embodiment of the first to fourth aspects.

### BRIEF DESCRIPTION OF THE FIGURES

FIG. 1 is a flowchart illustrating schematically a method of identifying a chain pair according to the disclosure.

FIG. 2 shows an embodiment of a system for identifying a chain pair according to the disclosure.

FIG. 3 shows the training procedure for an exemplary deep learning model as described herein. The method comprises obtaining unpaired antibody sequences, pretraining an encoder model using this data and a masked language modeling (MLM) task, combining the pretrained encoder (AntiBERTa) with cross-attention blocks, and using paired antibody sequences (true and random pairs) to fine-tune a model comprising the pretrained encoder and cross-attention blocks for prediction of a probability of belonging to the true pairs or random pairs category.

FIG. 4 shows in more detail the training procedure for an exemplary deep learning model as described on FIG. 3. A. Creation of the training, validation, and test sets for the masked language model task. B. The set up of the pre-training procedure, and how the “warmed up” model feeds into the subsequent step. C. Outline of how a warmed-up model can be used as part of a classifier to predict the likelihood of chain pairs forming functional chain pairings.

FIG. 5 illustrates schematically the task of mased language modelling for pretraining an encoder model to learn antibody chain sequence features (A), and the task of true vs random pair classification used for fine tuning of a classifier model comprising a pretrained encoder (B).

FIG. 6 illustrates schematically the architecture of a deep learning model as described herein (A) and the architecture of 3 cross-attention blocks used in such a model (B).

FIG. 7 shows results of evaluation of the classification performance of a deep learning model as described herein on an independent test data set comprising true pairs from single cell sequencing data and decoy random pairs generated from the same dataset. A. Receiver operator characteristic (ROC) curve. B. Precision-Recall (PR) curve.

FIG. 8 illustrates schematically the architecture of a heavy chain and a light chain. A. Architecture of an Ig heavy chain. The approximate boundaries of the V, D, and J genes are marked, along with the boundaries of the junction. A segment of the V-gene toward the N-terminus is in a dotted boundary as the read length from many NGS methods are too short to cover this region and/or some primers used for NGS are slightly inset within the V region. However, it is still possible to infer the V-gene using the sequence within the solid boundaries. B. Same as A., but for the light chain.

FIG. 9 illustrates schematically the training of an exemplary deep learning model as described herein. A. Pretraining of a generative transformer model (FAbCon) using a next toke prediction task. B. Architecture of the FabCon-small model. C. Fine tuning of the FabCon model for identifying chain pairs. D. Architecture of a deep learning model as described herein for identifying chain pairs.

### DETAILED DESCRIPTION OF THE INVENTION

In describing the present invention, the following terms will be employed, and are intended to be defined as indicated below.

A B cell receptor is a transmembrane protein expressed on the surface of B cells. A B cell receptor comprises a binding moiety (also referred to as “antigen-binding subunit” or “membrane immunoglobulin”, “mlg”) comprising a membrane bound immunoglobulin molecule (also referred to as antibody) that recognises a cognate antigen, and a signal transduction moiety. The membrane-bound immunoglobulin molecule comprises two immunoglobulin light chains and two immunoglobulin heavy chains, and is identical to a corresponding secreted antibody with the exception of an integral membrane domain. The signal transduction moiety is a heterodimer called Ig-α/Ig-β (CD79), bound together and to the immunoglobulin by disulfide bridges. An antibody (Ab) or immunoglobulin (Ig) is an immune protein, comprising an antigen binding site and a constant region belonging to one of a limited set of isotypes (IgA, IgD, IgE, IgG, or IgM) and mediating interactions with other components of the immune system. In humans and most mammals, antibodies comprise four polypeptide chains: two identical heavy chains and two identical light chains connected by disulfide bonds. Light chains typically consist of one variable domain V_{L} and one constant domain CL, while heavy chains typically contain one variable domain V_{H} and three to four constant domains C_{H}1, C_{H}2, . . . . The variable domains form the antigen binding region and can also be referred to as the F_{V} region.

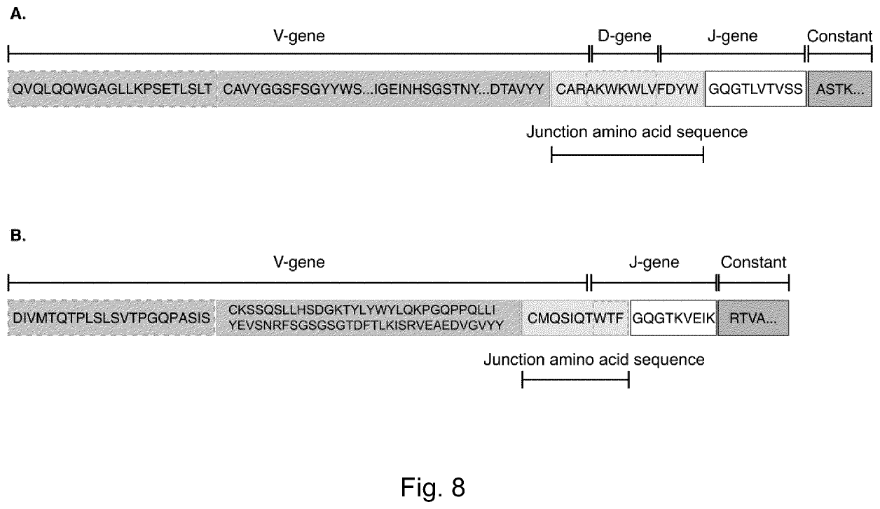

Each variable domain contains three hypervariable regions referred to as the complementarity-determining regions (CDRs), which together form an antigen binding site. The variable region of each immunoglobulin heavy or light chain is encoded in several pieces-known as gene segments (subgenes): Ig heavy chains comprise variable (V), diversity (D) and joining (J) segments, and Ig light chains comprise V and J segments. Multiple copies of the V, D and J gene segments are present in the genome and developing B cells assemble an Ig variable region by (nearly) randomly selecting and combining one V, one D and one J gene segment (or one V and one J segment in the light chain), in a process called V(D)J recombination. The process involves the formation of double-strand breaks between the required segments, which form hairpin loops that are then joined together. The joining process is inaccurate, resulting in the variable addition or subtraction of nucleotides between the V and J (light chain) or V and DJ and D and J (heavy chain) segments, producing a large diversity in the sequences at the junction between segments (referred to as “junction sequences”). The V(D)J recombination process produces novel amino-acid sequences in the antigen-binding regions of Igs, generating a vast diversity of antigen recognition capability. As a result of the process, each Ig heavy chain variable region comprises: a V segment, a D segment and a J segment, with a junction sequence that spans the join between these segments (as illustrated on FIG. 8A). Similarly, each Ig light chain hypervariable region comprises: a V segment, and a J segment, and a junction sequence that spans the join between these segments (as illustrated on FIG. 8B). Within the variable region, CDR1 and CDR2 are found in the V segment, and CDR3 includes some of the V, all of D (in the heavy chain) and some of the J segment.

A T cell receptor is a membrane anchored protein expressed on the surface of T cells. A T cell receptor comprises a pair of protein chains that together form binding moiety that recognises a cognate antigen. These are expressed in a complex with constant T cell coreceptor chains CD3, comprising a CD3γ chain, a CD3δ chain, and two CD3ε chains in mammals. The constant chains associate with the T cell receptor and the constant ξ-chain to form the TCR complex, which together is able to generate a signal upon antigen binding to the T cell receptor. The TCR is a heterodimeric protein, comprising two highly variable chains, the α and β chains (in the majority of T cells), or the alternative γ and δ chains (in a minority of T cells). Each chain comprises two extracellular domains: a variable region (or variable domain) and a constant region (or constant domain, proximal to the cell membrane), a transmembrane region and a short cytoplasmic tail. The variable regions together bind to a peptide (antigen), within the context of a MHC (major histocompatibility complex) molecule in the case of αβ TCRs. Each variable domain contains three hypervariable regions referred to as the complementarity-determining regions (CDRs, respectively referred to as CDR1, CDR2 and CDR3 on each of the chains), which together form an antigen binding site. The TCR is a member of the immunoglobulin superfamily, which comprises BCRs and antibodies. In a process similar to that explained above, the variable region of each TCR chain is encoded in several pieces-known as gene segments (subgenes): β and δ chains comprise variable (V), diversity (D) and joining (J) segments, and a and γ chains comprise V and J segments. Multiple copies of the V, D and J gene segments are present in the genome and developing T cells assemble a TCR chain variable region by (nearly) randomly selecting and combining one V, one D and one J gene segment (or one V and one J segment in the α/γ chain), in a process called V(D)J recombination. The process involves the formation of double-strand breaks between the required segments, which form hairpin loops that are then joined together. The joining process is inaccurate, resulting in the variable addition or subtraction of nucleotides between the V and J (α/γ chain) or V and DJ and D and J (β/δ chain) segments, producing a large diversity in the sequences at the junction between segments (referred to as “junction sequences”). The V(D)J recombination process produces novel amino-acid sequences in the antigen-binding regions of TCRs, generating a vast diversity of antigen recognition capability. As a result of the process, each β/δ chain variable region comprises: a V segment, a D segment and a J segment, with a junction sequence that spans the join between these segments. Similarly, each α/γ chain hypervariable region comprises: a V segment, and a J segment, and a junction sequence that spans the join between these segments. Within the variable region, CDR1 and CDR2 are found in the V segment, and CDR3 includes some of the V, all of D (in the heavy chain) and some of the J segment.

As used herein, a “variable chain” (also referred to herein simply as “chain”) of an antigen-binding protein refers to a chain of an antigen-binding protein that is involved in antigen recognition, or a part thereof that contains at least part of a variable region of the chain. Variable chains comprise variable regions that are responsible for the diverse repertoire of antigen recognition properties within antigen-binding proteins. A variable chain may be a BCR heavy or light chain, an antibody heavy or light chain, a TCR α or β chain, a TCR γ or δ chain, or any part of such chains that contains at least a part of one or more variable regions within these chains.

The B cell receptor repertoire (or corresponding antibody repertoire) present in a sample can be investigated using sequencing approaches. As explained above, two main sequencing approaches are used: single B cell sequencing, and sequencing of bulk B cell populations. As the BCR signalling moiety and antigen-binding moiety transmembrane domain is not variable, these techniques focus on the parts that are common between the B cell repertoire and the corresponding antibody repertoire. Thus, in the context of this disclosure, references to a BCR sequence, BCR repertoire, BCR heavy chain sequence, BCR light chain sequence, and any parts thereof, are used interchangeably with the corresponding antibody sequence, antibody repertoire, antibody heavy chain sequence, antibody light chain sequence, and corresponding parts thereof. For example, reference to sequencing a BCR heavy chain variable region equally is equivalent to sequencing the corresponding antibody heavy chain variable region, and the two terms may be used interchangeably. The term “antigen-binding protein” is used herein to refer to a BCR protein, a TCR protein, an antigen-binding moiety of a BCR protein, an antibody, or any parts thereof that maintain the antigen-binding property of the original BCR protein. TCR protein or antibody. Note that the repertoire of antibodies circulating in the blood of an individual may not match the B cell receptor repertoire present in the sample at the same time point. This is because antibodies that have been produced by B cells that are no longer present in the individual (e.g. because they have died) may be present in the sample. Thus, the term “corresponding antibody repertoire” refers to the repertoire of antibodies that would be expressed by the B cells present in a sample, not the repertoire of antibodies (proteins) that are actually present in the sample.

Single B cell sequencing can maintain the correspondence between heavy and light chain sequences. Two main approaches can be used to do this. The first approach is physical linkage of VH and VL [DeKosky et al., 2016]. The second approach is cell barcoding (such as e.g. provided by 10× Genomics) [King et al., 2021]. The physical linkage approach has a higher throughput than the cell barcoding approach, but it is more difficult to recover the full sequence. By contrast, cell barcoding has a lower throughput but allows easier recovery of the full sequence. No matter the approach, single B cell sequencing is limited in terms of throughput (to various extents), as explained above. Some single B cell sequencing technologies are additionally limited in terms of the length of the sequences recovered. As a result, BCR/antibody sequences identified using some single B cell sequencing methods may be limited to investigating a single CDR region, for example CDR3 (in other words, although the flanking V and J segments may be identified, they may not be fully sequenced to obtain the sequence of the CDR1 and CDR2 in the V segment), in both the heavy and light chain. In other words, datasets from single B cell sequencing methods may vary in the extent to which the sequence of the heavy and light chain is identified. Within the regions sequenced, it may also not be practical to sequence (or record) every single base of the V(D)J segments and as such sequencing efforts may focus on obtaining the junction sequence and enough information to identify the V, D and J genes. As a result, such methods may provide information comprising: the identity of the V, D and J segments (e.g. in the form of a V-/D-/J-gene segment identifier) for the heavy chain, the sequence of the junction segment in the heavy chain, the identity of the V and J segments (e.g. in the form of a V-/J-gene identifier) for the light chain, and the sequence of the junction segment in the light chain. The identity of the respective segments can be used to recover the corresponding germline sequence from a database. However, in cases where the data only contains the identity of the respective segments, any mutation that may be present in a particular chain (e.g. somatic mutations) compared to the reference germline sequence may not be captured. By contrast, sequencing of bulk B cell populations does not maintain the pairing between heavy and light chain sequences, but is less limited in terms of sequencing capabilities (in particular depth of sequencing of the BCR repertoire) within the heavy chain and light chain repertoires, respectively. Sequencing of bulk B cell populations may comprise sequencing of the heavy chain repertoire, the light chain repertoire, or both, of a B cell population. However, as mentioned above, due to the bulk nature of the process, even when both the light and heavy chain repertoires are sequenced, it is not possible to maintain the pairing information during the sequencing process. Such sequencing may produce information as sparse as that obtained with single cell B sequencing, or more detailed information including e.g. full CDR sequences, sequences of multiple CDRs, full variable region sequences, or full variable region sequences and enough of the constant region to determine the isotype of the sequence. Similar considerations apply to the sequencing of the T cell repertoire. In particular, many of the processes and limitations described above in relation to the study of B cell receptors and antibodies (and in particular in relation to the sequencing of these repertoires) apply to the T cell repertoire.

As used herein, the terms “variable chain sequence”, encompasses the terms “heavy chain sequence”, “light chain sequence”, “α chain sequence”, “β chain sequence”, “γ chain sequence” and “δ chain sequence” and refer to any information that can be obtained from B cell sequencing or T cell sequencing technologies, ranging from a combination of one or more gene segment identifiers and/or junction sequences at one end, to full chain sequences at the other end. In particular, the terms “heavy chain sequence” and “light chain sequence” refer to any information that can be obtained from B cell sequencing technologies, ranging from a combination of one or more gene segment identifiers and/or junction sequences at one end, to full chain sequences at the other end. Further, the terms “variable chain sequence”, “heavy chain sequence”, “light chain sequence” “α chain sequence”, “β chain sequence”, “γ chain sequence” and “δ chain sequence” refer interchangeably to the amino acid sequence or the corresponding nucleic acid coding sequence. Similarly, a variable chain pairing or pair (such as heavy-light chain pairing or pair) refers to a combination of a heavy chain sequence and a light chain sequence, an α chain sequence and a β chain sequence, or a γ chain sequence and a δ chain sequence as defined herein, each ranging from a combination of one or more gene segment identifiers and/or junction sequences at one end, to full chain sequences at the other end.

Within the context of providing a desired antibody or antigen-binding protein, such as e.g. a therapeutic antibody, the term “antibody” (Ab) includes monoclonal antibodies, polyclonal antibodies, multispecific antibodies (e.g., bispecific antibodies), and antibody fragments (e.g., scFv) that exhibit the desired biological activity and that comprise a heavy-light chain pairing identified as described herein or a heavy-light chain pairing derived from a heavy-light chain pairing identified as described herein (for example by further optimisation, affinity maturation, etc.).

A “sample” as used herein may be a cell or tissue sample, a biological fluid, an extract (e.g. a DNA or RNA extract obtained from the subject), from which B cell genomic material (e.g. RNA or DNA) can be obtained for genomic analysis, such as by sequencing (e.g. whole genome sequencing, whole exome sequencing, targeted/capture sequencing, RNA-seq, etc.). The sample may be a cell, tissue or biological fluid sample obtained from a subject (e.g. a biopsy). Such samples may be referred to as “subject samples”. In particular, the sample may be a blood sample, a lymph node sample, a spleen sample, or a tumour sample, or a sample derived therefrom (such as e.g. by B cell purification, T cell purification, RNA extraction, etc.). As used herein, the terms “genomic material”, “genomic sequencing” and the like encompasses both to the material/sequence present in the genome and the transcriptome of a sample, unless context indicates otherwise. The sample may be one which has been freshly obtained from a subject or may be one which has been processed and/or stored prior to genomic analysis (e.g. frozen, fixed or subjected to one or more purification, enrichment or extraction steps). The sample may be a cell or tissue culture sample. As such, a sample as described herein may refer to any type of sample comprising B cells or genomic material derived therefrom, whether from a biological sample obtained from a subject, or from a sample obtained from e.g. a cell line. The sample is preferably from a mammalian (such as e.g. a mammalian cell sample or a sample from a mammalian subject, such as a cat, dog, horse, donkey, sheep, pig, goat, cow, mouse, rat, rabbit or guinea pig), preferably from a human (such as e.g. a human cell sample or a sample from a human subject). Further, the sample may be transported and/or stored, and collection may take place at a location remote from the sequence data acquisition (e.g. sequencing) location, and/or any computer-implemented method steps described herein may take place at a location remote from the sample collection location and/or remote from the genomic data acquisition (e.g. sequencing) location (e.g. the computer-implemented method steps may be performed by means of a networked computer, such as by means of a “cloud” provider).

The term “sequence data” refers to information that is indicative of the presence of genomic material (DNA or RNA) or proteomic material in a sample that has a particular sequence. Thus, sequence data may comprise one or more nucleotide sequences and/or one or more amino acid sequences. Such information may be obtained using sequencing technologies, such as e.g. next generation sequencing (NGS), for example whole exome sequencing (WES), whole genome sequencing (WGS), whole transcriptome sequencing (RNAseq) or sequencing of captured genomic loci (targeted or panel sequencing). When NGS technologies are used, the sequence data may comprise a count of the number of sequencing reads that have a particular sequence. Sequence data may be mapped to a reference sequence, for example a reference genome, using methods known in the art (such as e.g. Bowtie (Langmead et al., 2009)). Thus, counts of sequencing reads or equivalent non-digital signals may be associated with a particular location or locus (where the “location” refers to a location in the reference genome or transcriptome to which the sequence data was mapped). Further, a location may contain a mutation, in which case counts of sequencing reads or equivalent non-digital signals may be associated with each of the possible variants (also referred to as “alleles”) at the particular location. The process of identifying the presence of a mutation at a particular location in a sample is referred to as “variant calling” and can be performed using methods known in the art (such as e.g. general purpose NGS variant callers such as the GATK HaplotypeCaller, gatk.broadinstitute.org/hc/en-us/articles/360037225632-HaplotypeCaller or tools specifically designed for immune sequences such as IgBLAST, www.ncbi.nlm.nih.gov/igblast/, [Ye et al.) Genomic sequence data may be converted to amino acid sequences by translating coding regions in silico (directly from an mRNA sequence or from identified coding regions in a genomic sequence), as known in the art.

As used herein “treatment” refers to reducing, alleviating or eliminating one or more symptoms of the disease which is being treated, relative to the symptoms prior to treatment. “Prevention” (or prophylaxis) refers to delaying or preventing the onset of the symptoms of the disease. Prevention may be absolute (such that no disease occurs) or may be effective only in some individuals or for a limited amount of time. A composition as described herein may be a pharmaceutical composition which additionally comprises a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

As used herein, the terms “computer system” includes the hardware, software and data storage devices for embodying a system or carrying out a method according to the above described embodiments. For example, a computer system may comprise a central processing unit (CPU), graphical processing unit (GPU), input means, output means and data storage, which may be embodied as one or more connected computing devices. Preferably the computer system has a display or comprises a computing device that has a display to provide a visual output display (for example in the design of the business process). The data storage may comprise RAM, disk drives or other computer readable media. The computer system may include a plurality of computing devices connected by a network and able to communicate with each other over that network. It is explicitly envisaged that computer system may consist of or comprise a cloud computer. The term “processor” encompasses any processing unit or combination of processing units, including in particular CPUs and GPUs. As used herein, the term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

### Identification of Variable Chain Pairs

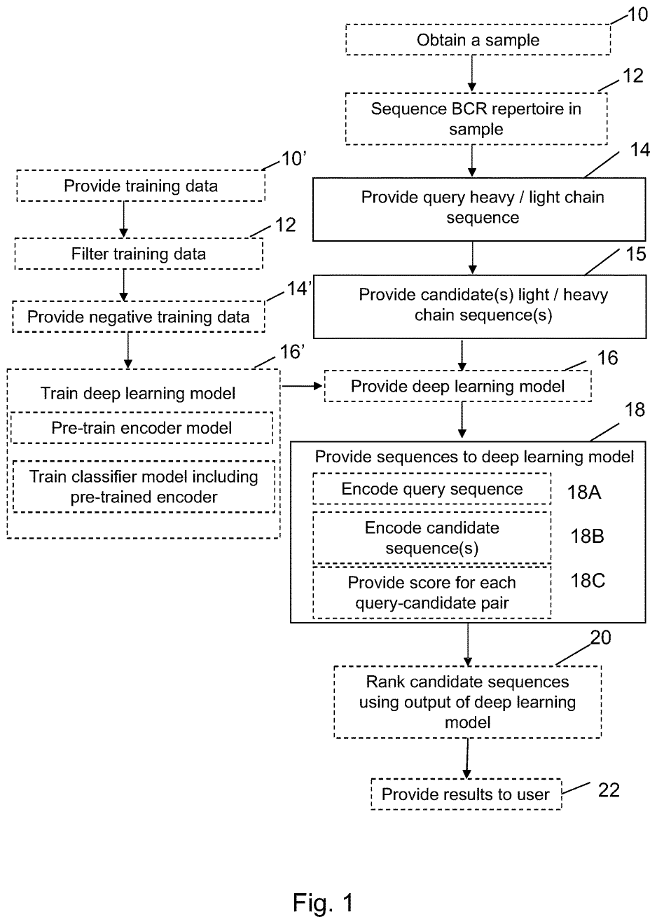

The present disclosure provides methods for identifying functional variable chain pairs and/or predicting whether a variable chain pair is likely to be functional. An illustrative method will be described by reference to FIG. 1. FIG. 1 illustrates an embodiment in which a heavy or light chain sequence of a B cell receptor or antibody is used to identify heavy-light chain pairs. In other words, FIG. 1 illustrates an embodiment in which the variable chain sequences are BCR/antibody heavy and light chains from BCR. However, the method described by reference to FIG. 1 is applicable to embodiments in which a TCR α, β, γ, or δ chain sequence is used to identify αβ (if the query chain or chain pair is an α or β chain or αβ chain pair) or γδ (if the query chain or chain pair is a γ or δ chain or γδ chain pair) chain pairs. At optional step 10, a sample comprising B cell genomic material (typically in the form of RNA, where the RNA encoding for the BCR expressed by the cells from which the B cell genomic material originated can be extracted and sequenced) may be obtained from a subject. Similarly, a sample comprising T cell genomic material may be used in embodiments where TCR chain pairs are identified. At step optional step 12, the BCR repertoire in the sample may be sequenced using bulk BCR sequencing. This may comprise sequencing the heavy chain BCR repertoire in the sample and/or the light chain BCR repertoire in the sample. Similarly, the TCR repertoire in the sample may be sequenced using bulk TCR sequencing. This may comprise sequencing the β chain repertoire and/or the α chain repertoire of in the sample. At step 14, a query chain sequence or sequence pair is provided. In the illustrated embodiment, the query sequence is a heavy chain sequence. In other embodiments, the query chain sequence may be a light chain sequence. In other embodiments, the query may be a chain sequence pair. Providing a query sequence may comprise selecting at step 14A a query sequence as one of the heavy chain sequences sequenced at step 12. Providing a query sequence pair may comprise selecting at step 14A a query sequence as one of the heavy chain sequences sequenced at step 12 and a query sequence as one of the light chain sequences sequenced at step 12 or a light chain sequence obtained from a database or other source. Providing a query sequence or sequence pair may comprise providing at step 14B a sequence that comprises (or pair of sequences that each comprise) a V-gene sequence or identifier, a J-gene sequence or identifier, and a junction sequence. For example, step 14B may comprise extracting, from a bulk BCR sequencing data set, for a selected sequence (or each of a pair of sequences), a V-gene sequence or identifier, a J-gene sequence or identifier, and a junction sequence. Similar steps may be performed in the context of TCR pairing, for example using a query β chain sequence. When a single query sequence is provided, step 14 may comprise step 14C of selecting one or more candidate sequences for pairing with the query sequence. These may be obtained from a database, computing device (including e.g. by simulation), or user interface. At optional step 16, a deep learning model is provided, wherein the deep learning model is configured to take as input a query pair of variable chain sequences (which may comprise a query sequence and a candidate sequence for pairing, or a query pair of sequences) and to produce as output a score indicative of the probability that the pair of sequence will form a functional pair. In the illustrated embodiment, the query sequence pair is a heavy-light chain sequence pair and thus the deep learning model is configured to take as input a query heavy chain sequence and a query light chain sequence and to produce as output a score indicative of the probability that the query heavy chain sequence and the query light chain sequence will form a functional pair. In other embodiments, the query chain sequence may be a heavy chain sequence and thus the deep learning model may be configured to take as input a query heavy chain sequence and a plurality of candidate light chain sequence and to produce as output a respective score indicative of the probability that the query heavy chain sequence and each of the candidate light chain sequences will form a functional pair, or information derived therefrom such as e.g. a ranking of the plurality of pairs or plurality of candidate light chain sequences based on their respective scores. In yet other embodiments, the query chain sequence may be a β chain sequence (or an α, δ or γ chain sequence) and thus the deep learning model may be configured to take as input a query β chain sequence (or an α, δ or γ chain sequence) and one or more candidate α chain sequence (or β, γ or δ chain sequence) and to produce as output a score indicative of the probability that the query β chain sequence and each of the candidate α chain sequences (or each of the corresponding candidate β, γ or δ chain sequences) will form a functional pair, or information derived therefrom (e.g. a ranking of a plurality of candidate pairs). The deep learning model may have been previously trained using training variable chain sequences from known variable chain pairs, such as training heavy and light chain sequences from known heavy-light chain pairs in the illustrated embodiment. Thus, providing a deep learning model may simply comprise retrieving a trained deep learning model from a computer-readable medium such as a memory associated with a processor executing the method, or otherwise receiving the trained deep learning model. The training of the deep learning model is explained in more detail below. Alternatively, the deep learning model may be at least partially trained as part of the present method, using training variable chain sequences from known heavy-light chain pairs, such as training heavy and light chain sequences from known heavy-light chain pairs in the illustrated embodiment, and optionally using training variable chain sequences from known unpaired heavy and light chains.

At step 18, a pair of sequences comprising a query chain sequence and a candidate corresponding chain sequence, or a query chain sequence pair, is provided to the deep learning model. Step 18 may comprise optional step 18A of encoding the sequences in the pair of sequences using a predetermined encoding scheme. The encoding scheme(s) used may have been previously defined based on the content of the training variable chain sequences (e.g. heavy and light) chain sequences used to train the deep learning model. Step 18 may comprise optional step 18B of selecting a sequence pair that is associated with the highest probability of forming a functional pair amongst a plurality of pairs of sequences (e.g. the highest score/highest probability or highest ranking). At optional step 20, the results of any of the preceding steps (and in particular step 18) may be provided to a user, for example through a user interface. These results may be used for example to provide a therapeutic antibody, as will be described further below. The method may be repeated for a plurality of query sequences. This may comprise repeating steps 14 to 18.

The deep learning model comprises an encoder module. An encoder module refers to a machine learning model that has been trained to take as input sequence data (protein chain sequences) and produce as output an embedded representation of the sequence data, or to produce as output a decoded or generated sequence from a learned embedded representation of the sequence data. Thus, a sequence encoder may be an encoder model of a bidirectional encoder only model, an encoder model of an encoder-decoder model, or a decoder model of a decoder only model such as a generative model. For example, generative models with architectures such as those of the LlaMa (Touvron et al., 2023), Falcon LLM (falconllm.tii.ae/), and GPT (e.g. GPT-3, Brown et al. 2020) may be used. Alternatively, transformer encoder-based architectures such as AntiBERTa (see Examples and Leem et al. 2022) may be used. When using a decoder model, the embedded representation may be obtained as the embeddings from the final decoder layer of the decoder. An encoder module may also be referred to herein as a “encoder”, although its architecture is not limited to that of an encoder. The embedded representation is a representation in latent space, typically configured such that the original sequence data can be reconstructed from the embedded representation. An encoder module is a deep learning model. An encoder module may be trained as part of a sequence-to-sequence model. An encoder module may be a transformer-based encoder or decoder. A deep learning model comprising the sequence encoding module may have been trained using a masked language modelling (MLM) task or a causal language modelling (CLM, next token prediction) task. The former may be used in particular for encoder only and encoder-decoder models, and the latter may be used in particular for generative models.

The training of the deep learning model will now be explained by reference to optional steps 10′-16′. At step 10′, training data is provided comprising at least training variable chain sequences from known variable chain pairs. In the illustrated embodiment, the training data comprises heavy and light chain sequences from known heavy-light chain pairs. The training data may comprise at least 20,000 training chain pairs, at least 30,000, at least 40,000, at least 50,000, at least 60,000, at least 70,000, at least 80,000, at least 90,000, at least 100,000, at least 120,000 or at least 150,000 training chain pairs. In embodiments related to B cell receptors/antibodies, the training data may comprise at least 80,000, at least 100,000, at least 120,000 or at least 150,000 pairs of training heavy and light chain sequences. In embodiments, the training data comprises at least 1, 1.5 or 2 million paired sequences. The training data may further comprise unpaired training sequences, which are heavy and light chain sequences in the illustrated embodiment (but can be any first and second chains as described herein). The unpaired training chain sequences may be referred to as “pre-training data”. In such cases, the paired training data may be referred to as “fine-tuning data”. In embodiments, paired training data is also used for pretraining. Thus, the training data may comprise training data for fine-tuning (comprising paired chain sequences, in particular paired heavy and light chain sequences in the illustrated embodiment), and pre-training data (comprising unpaired chain sequences, in particular unpaired heavy and light chain sequences in the illustrated embodiment, and optionally also paired training sequences). The pre-training data may comprise at least 100,000, at least 200,000, at least 300,000, at least 400,000, at least 500,000, at least 600,000, at least 700,000, at least 800,000, at least 900,000, at least 1 million (or at least 5, 10, 15, 20, 25, 30, 35 or 40 million) unpaired training chain sequences of the first type and/or of the corresponding type. In embodiments, the training data comprises at least 500 million, 600 million or 700 million individual sequences. The training data may comprise at least 1 million (or at least 5, 10, 15, 20, 25, 30, 35 or 40 million) unpaired training heavy chain sequences and at least 1 million (or at least 5, 10 or 15 million) unpaired training light chain sequences. As the skilled person understands, the amount of training and/or pretraining data may be limited by the amount of suitable data available, and may change as more data becomes available. The pre-training and/or training data may comprise simulated data. Simulated data may comprise paired or unpaired protein sequences obtained using methods for simulating antigen-binding protein sequences, such as e.g. immuneSIM (Weber et al., 2020) and/or ProGen2 (Nijkamp et al., 2022). The pretraining data may be collected and/or used for pretraining as described in Leem et al., 2022. For example, in Leem et al., 2022, unpaired chain sequences comprising approximately 42 million heavy chains and 15 million light chains were used. More data may advantageously be used if available. Further, the amount of data available may depend on the particular use case, such as e.g. the identity of the first and corresponding chain sequences (e.g. more data may be available for αβ TCRs than for γδ TCRs which are rarer), the criteria used when filtering the data (see step 12′) etc. The numbers provided may apply to the data prior to and/or after any filtering is applied. At step 12′, the training data is filtered. For example, the training data may be filtered to exclude any chain that comprises a number of amino acids within any predetermined region of the chain that is outside of a predetermined range of lengths. For example, the pre-training data may be filtered to exclude chain sequences that do not comprise at least 20 amino acids before the CDR1 region, sequences that do not comprise at least 10 amino acids after the junction sequence, sequences that do not comprise between 5 and 12 residues in the CDR1 region, sequences that do not comprise between 1 and 10 residues in the CDR2 region, and/or sequences that do not comprise between 5 and 38 residues in the CDR3 region. As another example, the training data may be filtered based on any feature of the data, including for example the cell type that the data was derived from, the organism, whether the data is from a naïve library, whether the data is from subjects that have been immunised with a particular antigen, etc. In other words, the training data may be filtered to ensure that the training data only contains (inclusion filter) or does not contain (exclusion filter) data with one or more features of interest. At step 14′, negative training data may be provided comprising non-native pairs of chains, also referred to as “negative training data”. The non-native pairs of chains may be obtained by randomly reassigning chains within pairs in a paired data set. Alternatively, the non-native pairs of chains may be obtained by randomly pairing chains from one or more unpaired data sets comprising both types of chains (e.g. unpaired heavy chains and unpaired light chains). For example, heavy chains from bulk heavy chain sequencing data may be randomly paired with light chains from bulk light chain sequencing data. Instead or in addition to the use of randomly created negative pairs, negative pairs may be selected as pairs that are known to not be functional, for example from prior experiments Pairs that are known not to be functional may include one or more of: pairs that could not be expressed in one or more prior experiments, pairs that did not have one or more desired functional characteristics in one or more prior experiments (e.g. lack of binding to target, etc.)

At step 16′, the training data is used to train a deep learning model to take as input a query pair comprising a heavy chain sequence and a light chain sequence (in the illustrated embodiment) and to produce as output a score indicative of the probability that the pair is functional (such as e.g. a probability that the pair is functional, in the illustrated embodiment) or information derived therefrom (such as e.g. a ranking of a plurality of input pairs based on a score indicative of the probability that each pair is functional), using the training data. Training the deep learning model may comprise first training a model comprising the encoder module, for example an encoder-decoder-based model or a bidirectional encoder model (also referred to as “sequence-to-sequence” model) or a decoder based model (e.g. a generative language model) using the unpaired training chain sequences to obtain a pretrained encoder model, and using the pre-trained model to initialise the encoder module of a deep learning model comprising an encoder module and a classification module which is trained for paired sequence prediction (fine-tuning). Alternatively, training the deep learning model may comprise training a first and second sequence-to-sequence model using the unpaired training chain sequences of the first type and of the second type, respectively (the first type being the heavy chain and the second type being the light chain, in the illustrated embodiment), and using the pre-trained encoder modules of these respective models to initialise respective encoder modules of the deep learning model. Alternatively, training the deep learning model may comprise training a sequence-to-sequence or generative language model using the concatenated training chain sequences of the first type and of the second type, (the first type being the heavy chain and the second type being the light chain, in the illustrated embodiment), and using the pre-trained encoder module of such a model (which may be an encoder or a decoder, depending on the model architecture) to initialise an encoder module of the deep learning model taking as input a concatenated pair of sequences. Training the deep learning model may comprise obtaining training sequences that comprise full length sequences for the variable region of the second type of chain and/or the first type of chain (e.g. the light chain and/or the heavy chain, in the illustrated embodiment) by imputing missing sequence information, if the chain sequences from known chain pairs do not comprise full length sequences for said variable regions. Training of a sequence-to-sequence model may comprise training the model using MLM. Training of a generative language model may comprise training the model using CLM. Step 16′ may comprise defining one or more encoding schemes for the training data by obtaining a vocabulary for encoding of the training chain sequences, in particular for encoding of the training heavy chain sequences and a vocabulary for encoding of the training light chain sequences in the illustrated embodiment. The encoding scheme may encode every single amino acid as a separate token, or may encode subsets of sequences as individual tokens (such as e.g. complete regions, k-mers within regions or using byte pair encoding). Defining an encoding scheme may comprise excluding from the vocabulary constructed based on the content of the training chain sequences any token that is used a number of times below a predetermined threshold (e.g. 2) in the training data. The present inventors have found encoding schemes that represent each amino acid as a token to be practical at least in the case of prediction antibody/BCR light-heavy chain pairings, where training data is available at sufficient sequence resolution and in sufficiently large amounts to constrain a model using such a granular representation.

### Systems

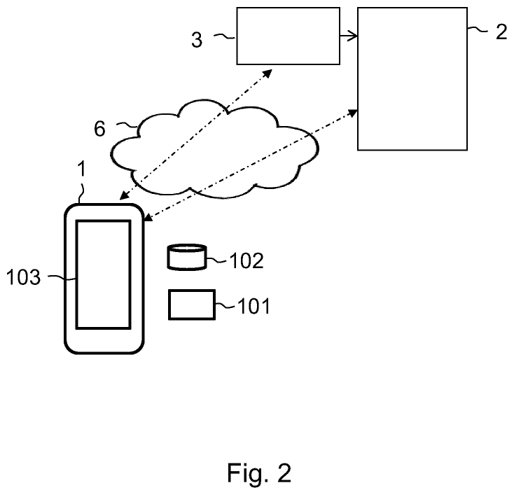

FIG. 2 shows an embodiment of a system for identifying a variable chain pair for an input variable chain, or predicting whether an input variable chain pair is likely to be functional, according to the present disclosure. The system comprises a computing device 1, which comprises a processor 101 and computer readable memory 102. In the embodiment shown, the computing device 1 also comprises a user interface 103, which is illustrated as a screen but may include any other means of conveying information to a user such as e.g. through audible or visual signals. The computing device 1 is communicably connected, such as e.g. through a network 6, to sequence data acquisition means 3, such as a sequencing machine, and/or to one or more databases 2 storing sequence data. The one or more databases may additionally store other types of information that may be used by the computing device 1, such as e.g. reference sequences, parameters, etc. The computing device may be a smartphone, server, tablet, personal computer or other computing device. The computing device is configured to implement a method for identifying a variable chain pair or predict whether a variable chain pair is likely to be functional (suitably a heavy-light chain pair or an αβ chain pair, advantageously a heavy-light chain pair), as described herein. In alternative embodiments, the computing device 1 is configured to communicate with a remote computing device (not shown), which is itself configured to implement a method of identifying a variable chain pair or predicting whether a variable chain pair is likely to be functional, as described herein. In such cases, the remote computing device may also be configured to send the result of the method to the computing device. Communication between the computing device 1 and the remote computing device may be through a wired or wireless connection, and may occur over a local or public network such as e.g. over the public internet or over WiFi. The sequence data acquisition 3 means may be in wired connection with the computing device 1, or may be able to communicate through a wireless connection, such as e.g. through a network 6, as illustrated. The connection between the computing device 1 and the sequence data acquisition means 3 may be direct or indirect (such as e.g. through a remote computer). The sequence data acquisition means 3 are configured to acquire sequence data from nucleic acid samples, for example genomic DNA samples or RNA samples extracted from B cells or T cells purified from fluid and/or tissue samples (such as e.g. peripheral blood, spleen, lymph node, tumour tissue, or any other type of sample comprising B cells or T cells). In some embodiments, the sample may have been subject to one or more preprocessing steps such as DNA/RNA purification, fragmentation, library preparation, target sequence capture (such as e.g. exon capture and/or panel sequence capture). Any sample preparation process that is suitable for use in the determination of a B cell receptor sequence or repertoire may be used within the context of the present invention. The sequence data acquisition means is preferably a next generation sequencer. The sequence data acquisition means 3 may be in direct or indirect connection with one or more databases 2, on which sequence data (raw or partially processed) may be stored.

### Applications

The above methods find applications in any context where it is desirable to identify an antibody or BCR that is likely to bind its target from information that is limited to the heavy chain, the light chain, unpaired heavy and light chains, or parts thereof (such as e.g. the V-gene, J-gene and junction sequences). This is frequently the case in the context of the discovery process of antibody therapeutics. Antibody therapeutics have been shown to be successful approaches for a wide range of diseases from neurodegenerative diseases to cancer. Thus, the approaches described herein find use in the context of providing therapeutics in each of these clinical contexts. Further, the methods described herein can be used to identify a potentially functional antibody or BCR from any input heavy/light chain or part thereof, whether the input information is newly generated for a particular purpose (e.g. from patients or samples identified as having a desired phenotype) or from existing/historical data sets (for example to mine or re-mine existing datasets to discover new therapies or identify immune proteins that could explain why certain clinical phenotypes persist).

Thus, the invention also provides a method of providing an antibody therapeutic, the method comprising identifying a heavy-light chain pairing using any of the methods described herein, or that is derived from a heavy-light chain pairing that has been identified using any of the methods described herein (such as e.g. by further optimisation, mutation, etc). The heavy-light chain pairing may be obtained for an input heavy chain sequence that has been obtained by bulk BCR sequencing of the heavy chain repertoire in one or more samples. The heavy-light chain pairing may be obtained for an input heavy chain sequence that has been obtained by bulk BCR sequencing of the heavy chain repertoire in one or more samples, and one or more candidate light chain sequences that have been obtained by bulk BCR sequencing of the light chain repertoire in one or more samples (where the one or more samples may be the same samples used to analyse the heavy chain BCR repertoire, or different samples). The one or more samples may be from one or more subjects. The one or more subjects may have been identified as having a desired characteristic, such as e.g. a particular clinical phenotype or clinically relevant characteristic such as a biomarker profile. For example, the one or more subjects may be resilient to a particular disease or condition. The disease or condition may be selected from a cancer (such as e.g. breast cancer), a neurodegenerative disease (such as e.g. amyotrophic lateral sclerosis), or an infectious disease (such as e.g. COVID-19). The method may comprise identifying a heavy-light chain pairing for a plurality of input heavy chain sequences selected from the heavy chain sequences identified in the one or more samples, thereby obtaining a set of heavy-light chain pairings. The method may further comprise identifying the target (or a putative target or sets of targets) of the heavy-light chain pairing or each heavy-light chain pairing in the set of heavy-light chain pairings. The method may further comprise identifying one or more targets by screening antibodies from the same source(s) as the one or more samples against a plurality of candidate peptides. The plurality of candidate peptides may be selected based on the species from which the one or more samples originate. For example, the source of the one or more samples may be one or more human subjects and the antibody repertoire(s) from the same source(s) as the one or more samples may be screened against a set of candidate peptides representative of the human peptidome.

Identifying the target (or a putative target or sets of targets) of the heavy-light chain pairing or each heavy-light chain pairing in the set of heavy-light chain pairings may comprise using one or more targets identified by screening antibodies from the same source(s) as the one or more samples against a plurality of candidate peptides. The method may further comprise filtering the set of heavy-light chain pairings based on one or more criteria. The one or more criteria may apply to the identity of the putative targets or sets of targets identified for a heavy-light chain pairing. The method may further comprise obtaining an antibody or fragment thereof which comprises an identified heavy-light chain pairing or a heavy-light chain pairing derived from an identified heavy-light chain pairing. Obtaining an antibody or fragment thereof may comprise identifying a coding sequence for the antibody or fragment thereof and expressing the sequence in a suitable expression system (such as e.g. in a suitable host cell). The method may further comprise identifying one or more antigens that the antibody or fragment thereof binds to, for example by testing for binding to one or more candidate antigens. The method may further comprise optimising the sequence of the antibody or fragment thereof. Optimising the sequence of the antibody or fragment thereof may be performed using any antibody optimisation technique known in the art. Optimising the sequence of the antibody or fragment thereof may be performed using information from the sequence data from which the heavy-light chain pairing was identified, for example by analysing sequences similar to the input sequence from which the heavy-light chain pairing was identified.

The invention also provides a method for providing an immunotherapeutic composition, the method comprising identifying a heavy-light chain pairing as described herein and producing an immunotherapeutic composition that comprises an antibody comprising the heavy-light chain pairing or an antibody that has been derived from the heavy-light chain pairing (such as e.g. by further optimisation, mutation, etc).

The methods described herein may also find uses in the context of providing bispecific antibodies. For example, the methods described herein may be used to identify a light chain that would be suitable for pairing with two different heavy chains of interest. Thus, the invention also provides a method of providing a bispecific antibody, the method comprising identifying a common light chain pairing for each of two heavy chains using any of the methods described herein, or a combination of a common light chain and two heavy chains that is derived from a heavy-light chain pairing that has been identified using any of the methods described herein (such as e.g. by further optimisation, mutation, etc). In such embodiments, it may be advantageous to use the deep learning model to predict a score indicative of the probability of functional pairing for a plurality of candidate light or heavy chain sequences for each query sequence. For example, the deep learning model may be used to predict a score/probability of a first query heavy chain forming a functional pair with each of a first plurality of candidate light chain sequences, and to predict a score/probability of a second query heavy chain sequence forming a functional pair with each of a second plurality of candidate light chain sequences. The first and second plurality of candidate light chain sequences may advantageously be the same or may at least partially overlap. The scores/probabilities predicted for the first and second plurality of light chain sequences may then be compared to identify one or more light chains that may be suitable for use as the common light chain of a bispecific antibody that includes both of the heavy chains. For example, a candidate light chain that has a relatively high probability of forming a functional pair with both heavy chains may be used. For example, candidate lights chains may be ranked based on the sum of the score/probability of forming a functional pair with the first heavy chain and the second heavy chain (or any other combined metric combining both probabilities).

The methods described herein may also find uses in the context of antibody optimisation. For example, the methods described herein may be used to identify heavy-light chain pairing that has one or more advantageous properties (such as e.g. improved functional or developability properties) compared to an original pairing for e.g. the heavy chain of the pairing. For example, the heavy chain of a pair may be paired with a plurality of candidate light chain pairs and a score indicative of the probability of the candidate forming a functional pair with the heavy chain may be determined. The candidate pairs may then be ranked or otherwise prioritised based on these scores/probabilities and optionally other criteria that apply to functional or developability properties. Thus, the invention also provides a method of providing an improved antibody, the method comprising identifying a heavy-light chain pairing using any of the methods described herein from an input heavy chain (or light chain) of an original antibody, or a heavy-light chain pairing that is derived from a heavy-light chain pairing that has been identified using any of the methods described herein (such as e.g. by further optimisation, mutation, etc). The methods described herein also find applications in any context where it is desirable to identify a TCR that is likely to bind its target from information that is limited to the β chain, the α chain (or, in less common cases, the γ or δ chain) or parts thereof (such as e.g. the V-gene, J-gene and junction sequences). This is frequently the case in the context of the discovery process of cell therapeutics such as engineered T cells. Thus, the invention also provides a method of providing a TCR-based therapeutic, such as an engineered T cell expressing a particular TCR, the method comprising identifying an αβ or γδ chain pairing using any of the methods described herein, or that is derived from an αβ or γδ chain pairing that has been identified using any of the methods described herein (such as e.g. by further optimisation, mutation, etc). Thus, the methods described herein may also find uses in the context of T cell receptor optimisation, in a similar way as described above for antibodies.

The following is presented by way of example and is not to be construed as a limitation to the scope of the claims.

### EXAMPLES

These examples describe a method of identifying heavy-light chain pairings and/or predicting the probability that a pair of chains will be functional, according to the present invention, and validate it using single-cell datasets with known pairings.

### Example 1—AntiBERTa for Pairing Prediction

### Methods

### Datasets

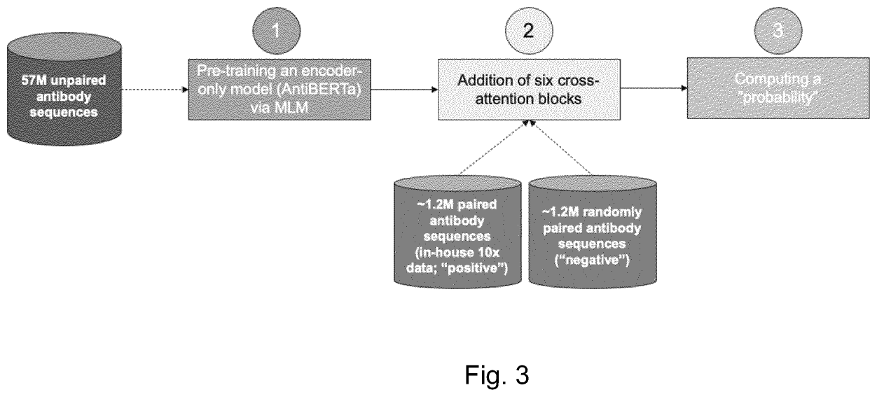

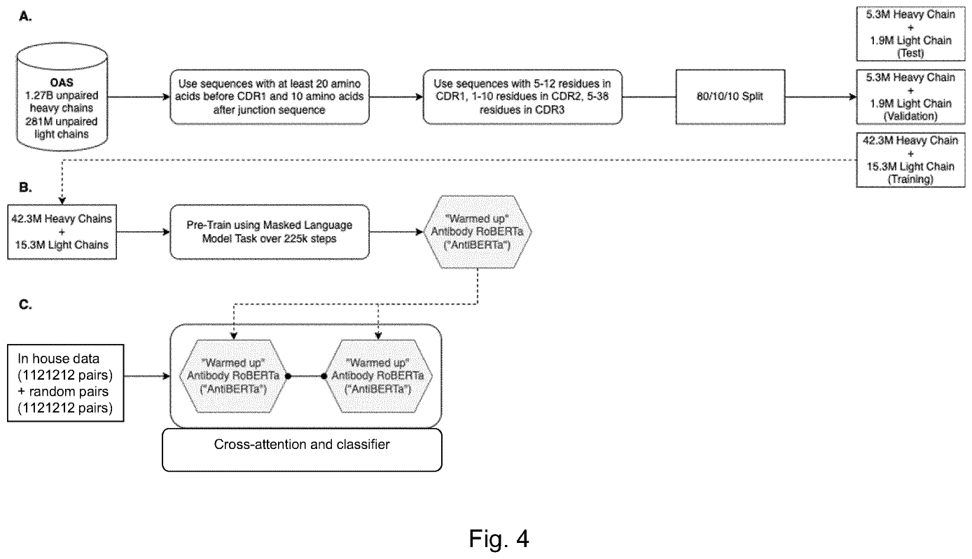

The model was trained in two steps (illustrated on FIGS. 3 and 4): a pretraining step in which a bi-directional transformer encoder model based on the ROBERTa architecture (Liu et al., 2019) was trained using unpaired heavy and light chain antibody sequences (step 1 on FIG. 3, steps A-B on FIG. 4); and a fine tuning step in which a model comprising the pretrained encoder of the transformer and a classifier module was trained using known pairs (positive examples) and random pairs (negative examples) (steps 2-3 on FIG. 3, step C on FIG. 4). The resulting model was then tested on independent test data. The following datasets were used. The pretraining step is described in Leem et al., 2022. The pretrained model is referred to as “AntiBERTa”. In Leem et al., 2022, the pretrained model was fine tuned for paratope prediction using human antibody sequences from the structural antibody database (SAbDab, Dunbar et al., 2014), In the present examples, the pretrained model was used directly for fine tuning for pairing of heavy-light chains as described below. However, it is also possible to use a fine-tuned model as described in Leem et al. 2022 (i.e fine tuned for paratope prediction) as a starting point for fine tuning for pairing as described herein.

Pretraining: human antibody sequences spanning 61 studies were downloaded from the OAS database (Kovaltsuk et al., 2018). Antibody sequences were first filtered out for any sequencing errors, as indicated by OAS. Sequences were also required to have at least 20 residues before the CDR1 and 10 residues following the CDR3. Finally, sequences were filtered to have 5-12 residues in the CDR1, 1-10 residues in the CDR2, and 5-38 residues in the CDR3. This results in a maximum sequence length of 148 residues. The entire collection of 71.98M unique sequences (52.89M unpaired heavy chains and 19.09M unpaired light chains) was then split into disjoint training, validation, and test sets using an 80:10:10 ratio. In total, the MLM training set comprised 42.3M heavy chain and 15.3M light chain sequences, while the MLM validation and MLM test sets each consist of 5.3M heavy chains and 1.9M light chains. AntiBERTa is a single model that is trained on both heavy and light chains.

Fine Tuning: Fine tuning was performed using an in-house dataset of single-cell B cell receptor sequencing data [referred to as the “Alchemab paired BCR dataset”]. These represent positive examples (known pairs). The classifier model needs a heavy and light chain, separately, as inputs. For training, the user must provide a number of 1 or 0, 1 denoting that the heavy and light chain are true, genuine pairs, and 0 denoting that the heavy and light chains are not true pairs. Since a “negative” dataset of VH-VL pairs is not available, this was approximated by randomly shuffling the Alchemab paired BCR dataset, and assuming that randomly generated pairs are not pairable. Thus, the classifier strictly speaking predicts whether a pair is likely to be a native pair or a random pair (the former being assumed to be functional and the latter being assumed to be non-functional or at least non-pairable). A total of 1121212 paired sequences were used as the “positive” set, and a further 1121212 were randomly paired to generate a “negative” set. Together, there are 2242424 pairs of sequences in total. Further quality control steps were applied to the set of 2242424 sequences (comprising both native/positive and random/negative pairs), in particular to remove sequences that: (i) are erroneously too long (using a cut-off identified as reasonable based on expert knowledge, set to 250 residues in the present example although other values may be equally suitable based on the particular data used), or (ii) have stop codons in the reads (identified as “*” in the amino acid sequences provided as output of the single cell sequencing analysis software—although information from the original RNA sequence reads may be used instead for this), or (iii) heavy chain V gene and light chain V gene pair occur too infrequently (fewer than 10 pairs in total). These criteria were applied to the combined set of native and random pairs.

After quality control, a total of 2239117 pairs remained (i.e. approx. 0.15% of the pairs were filtered out), of which 1791293 were used for training, 223912 for validation, and 223912 for testing. The pairs were randomly assigned to the training, validation and testing datasets. Other approaches are possible, such as e.g. sampling separately from the negative and positive pairs to ensure a balanced representation of the two categories in each data subset. The validation helps to choose a state of the neural network that should have optimal performance, and the test set is used for testing the network's generalisation performance.

Testing: A final independent evaluation of the performance of the model was performed using publicly available datasets of single-cell B cell receptor sequencing data (10×single-cell data) from King et al. 2021 (7 donors, 30222 unique heavy-light chain pairs), Eccles et al. 2020 (1 donor, 741 unique heavy-light chain pairs) and Setliff et al. 2019 (2 donors, 4944 unique heavy-light chain pairs). These were only used for final testing and therefore represent an unbiased evaluation of the performance of the model.

### Sequence Tokenisation

The pre-training data set contained full-length heavy chain and light chain sequences, and these were tokenised as single amino acids. The fine tuning and testing data also contained full-length heavy chain and light chain sequences, which were tokenised as single amino acids.

If any of the data to be used (whether for training, testing or use of the model) did not contain full-length heavy chain and light chain sequences, e.g. because the sequencing method used to generate this data was not able to recover the full amino acid sequence of the light/heavy chain, it would still be possible to use the model trained using full length sequences tokenised as single amino acids. For example, some datasets contain: (i) for each heavy chain: the V gene identifier, the junction sequence the J gene identifier, and the D gene identifier, and (ii) for each light chain: the V gene identifier, the junction sequence, and the J gene identifier. In such cases, each entry may comprise a combination of gene identifiers and sequences such as e.g. IGHV3-23/CAR . . . DYW/IGHJ6-IGKV3-20/CQQ . . . /IGKJ2. Such sequence can be converted to full amino acid sequences by imputing the germline sequences for the corresponding portions (e.g. the germline sequences of the corresponding V and J gene identifiers).

As mentioned above, in the present example all sequences were tokenised as single amino acids. The vocabulary used is composed of 25 tokens: the standard 20 amino acids and five special tokens (<s>, </s>, <pad>, <unk>, and <mask>). Each amino acid acts as a token, and no byte-pair encoding was used. Each chain is encoded with the start (<s>) and end (</s>) tokens; <pad> tokens are used to pad out tensors to the maximum sequence length of the mini-batch. <unk> tokens are used for ambiguous amino acids, such as X. A maximum length of 150 is allowed, as it covers the maximum sequence length in the pretraining dataset (148), along with the start and end tokens. Briefly the advantage of this is that it covers the training set in OAS, while ensuring that the amount of unnecessary padding is minimised.

Other tokenisation schemes could be used and are explicitly envisaged. For example, the models could be trained using tokenised sequences corresponding to a V gene identifier, a junction sequence and a J gene identifier. This may be particularly useful in cases where only partial sequence information is available, as explained above. In order to deal with this data format, a custom encoding method can be used, where each V-gene constitutes a single token, each J-gene constitutes a single token, and the junction amino acid sequence is tokenised as single amino acids as above. The junction sequence is the most diverse region of the sequence, and is believed to mediate most of the binding functionality, hence the increased granularity in tokenisation of this sequence. In such a scheme, tokens may be used if there was a minimum of e.g. 2 occurrences in the training set. Other possible schemes for the tokenisation of the junction amino acid sequences (or the full sequence) include for example byte-pair encoding, or tokens for overlapping k-mers (such as e.g. 3-mers). Schemes such as byte-pair encoding may be particularly useful if more full-sequence sequence data was available, such as e.g. if single-cell data in the order of hundreds of thousands, or even millions of sequences, was used for training the model. Using byte-pair encoding, several non-overlapping amino acids are encoded as tokens using a dictionary that is defined automatically in a data-driven manner. Byte pair encoding is a subword tokenisation scheme that replaces common pairs of consecutive bytes (in the present case, consecutive amino acids) with a byte that does not appear in the data. All pairs that occur more than once in the data are replaced by a corresponding token.

In the context of this example, a “sentence” is a tokenised representation of a heavy or light chain sequence. Each sentence starts with the special token <s>, followed by a token for each amino acid (or a token representing the heavy or light chain's V-gene, the overlapping 3-mer tokens, the J-gene token, or a token for each consecutive pair of amino acids), and then the special token </s>. For any sentence with fewer tokens than the maximum length (150 in the case of single amino acid encoding, 34 in the case of the custom encoding described above), the sequence was padded with the special <pad> token.

### Model

A bi-directional transformer model based on the ROBERTa architecture (Liu et al., 2019) was pre-trained, as described in Leem et al., 2022. The model was pre-trained using a dataset comprising both heavy and light chain sequences, as described above. Note that separate ROBERTa models could have been pre-trained on heavy and light chain sequences, respectively. This was not found to be necessary as the model was able to distinguish between the two types of chain and learn the features of a light and a heavy chain sequences separately. This pre-trained ROBERTa model trained on unpaired antibody sequences is referred to herein as “AntiBERTa”, and the training procedure is described in FIG. 4 (steps A and B).

Two copies of the AntiBERTa model (encoder only) were then used to process heavy and light chains, respectively, and the outputs of these were provided to a cross-attention module and a classification module, as described further below. The resulting model was fine-tuned using a dataset as described above (2242424 paired sequences, including 1121212 random pairs which form a negative set assigned a label of “0” and 1121212 real pairs which form the positive set assigned a label of “1”), step C on FIG. 4. Parameters were shared between the two AntiBERTa models in the fine tuning step (i.e. the two models are “Siamese” models). The use of pretrained so called “checkpoints” has been shown to be a powerful strategy in the context of NLP [Rothe et al., 2020]. In particular, a BERT-to-BERT architecture was shown to perform well for NMT in Rothe et al. [2020].

If the paired training data that was used to fine-tune the full model (including the two copies of the pre-trained AntiBERTa model) did not include full-length BCR heavy and light chain sequences, an equivalent paired data set that includes full-length sequences could be generated using one of two alternative approaches. In a first approach, a full-length sequence can be obtained by replacing the V and J gene identifiers with their corresponding germline sequences. In a second approach, the pre-trained AntiBERTa models (or any other such “checkpoint” model such as e.g. a GPT-2 model) can be used to predict the full-length sequence of the training set, independently for the heavy and light chains (using the respective models) based on the known parts of the chains. The prediction from the “checkpoint” models may be obtained using some or all of the known parts of the chain (e.g. gene segment identifiers, partial sequences etc.), optionally in combination with some information obtained from the germline sequences of any segment for which a full sequence is not available (such as e.g. the identity of some of the amino acids of the segment, for example the first k amino acids of the segment, where k can for example be 1, 2, 3, 5, 10, etc). In other words, the AntiBERTa model trained on the unpaired full-length heavy and light chain data may be used to predict the full-length sequence of the heavy chains in the training data from the V gene identifier, J gene identifier and junction sequence provided in the data. Similarly, the AntiBERTa model trained on the unpaired full-length heavy and light chain data may be used to predict the full-length sequence of the light chains in the training data from the V gene identifier, J gene identifier and junction sequence provided in the data. The same two approaches could be used to map any limited paired training data into data in a more extended format that may have been available to train the “checkpoint” models. Alternatively, the data used to train the “checkpoint” models may be converted to a limited format that matches the format of the paired training data. This may still benefit from the potential additional information gathered by the pre-trained models from the vast number of unpaired sequences available. However, it may not take full advantage of the extent of information available in such unpaired sequence data. In the present examples, full-length sequences were available both for pre-training and for fine-tuning, and this was therefore not necessary.

### Model Architecture-Pretraining

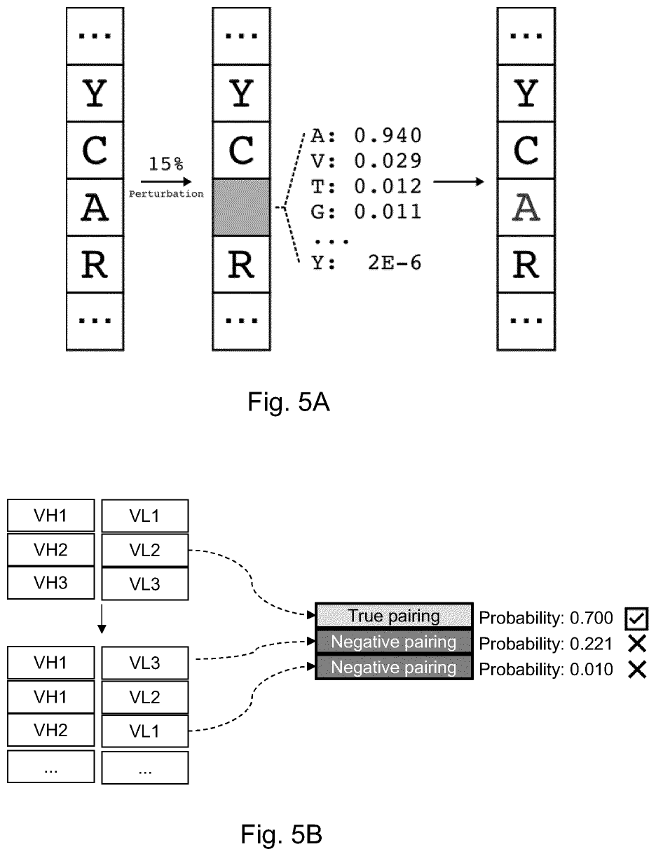

A 12-layer bi-directional transformer model based on the ROBERTa architecture (Liu et al., 2019) was pre-trained, as described in Leem et al., 2022. This model has an embedding dimension of 768, a feed-forward dimension of 3072, and 12 encoder layers and 12 self-attention heads. These are pre-defined accepted standards. In total, the model has 86 million learnable parameters. The model has a maximum sequence length of 150. It is first trained on a masked language modelling (MLM) task, which is akin to “filling in the blanks”. The MLM process used is illustrated schematically on FIG. 5A. During MLM pre-training, 15% of residues in an input sequence are perturbed, where the amino acid is replaced with a mask (grey square in FIG. 5A) in 80% of cases, the original amino acid in 10% of cases, and a random amino acid in the remaining 10% of cases (as described in Liu et al., 2019). The model is trained to predict the correct amino acid in in the perturbed positions, using a loss per sequence S={s1, s2, . . . sl} in a batch B with perturbed positions M provided by L_{MLM}=

$${L}_{M L M} = \frac{1}{\vert B \vert} {\sum }_{S ϵ B} {\sum }_{i ϵ M} \hat{p} ( {s}_{i} \vert {S}_{∖ M} ) .$$

For the MLM step, only unpaired sequences from the OAS dataset are used. For pre-training, the model was trained for a total of 225,000 steps, which includes a warm-up of 10000 steps up to a peak learning rate of 1e-4, linearly decayed thereafter. A batch size of 96 was used across eight NVIDIA V100 GPUs, with a global batch size of 768. The exact settings that are suitable for training a model are typically empirically determined depending on the details of the model and the training data, and many other values would likely be suitable. It is within the abilities of the skilled person to identify suitable settings. In the present examples, an encoder with a transformer architecture was used. However, alternative encoders may be used. For example, encoder modules of any encoder-decoder recurrent neural network model may be used, such as e.g. a Gated Recurrent Unit (GRU), using the same vocabulary as for the transformer, and also providing a latent representation of an amino acid chain. GRUs, and more broadly, other recurrent neural network (RNN) architectures such as LSTMs (long short term memory networks) with attention were the previous “state of the art” for neural machine translation before transformers became common for this task. GRU networks (and by extension, recurrent neural networks) have an entirely different mechanism of how they process sequences compared to Transformers. Briefly, transformers use a series of “self-attention” mechanisms that make them not only faster, but more accurate, while recurrent nets do not have this at all.

### Model Architecture—Fine Tuning

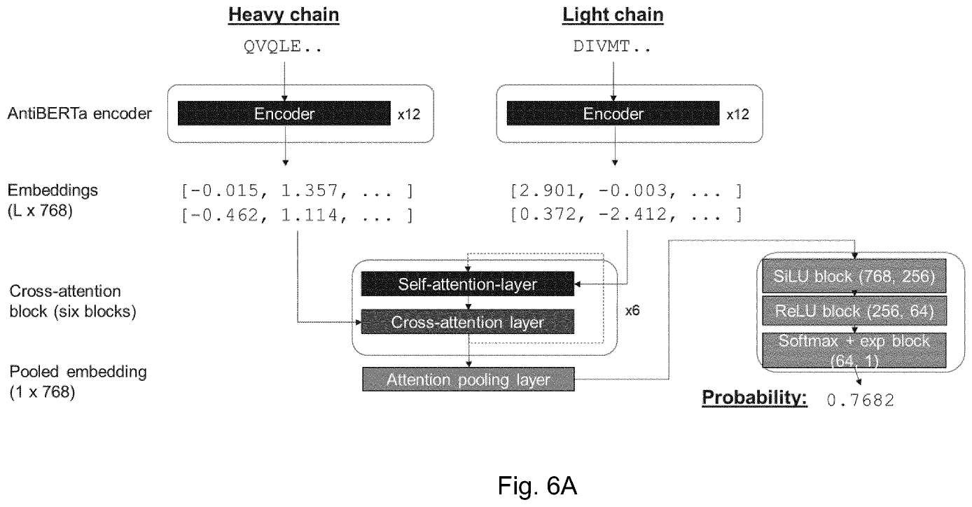

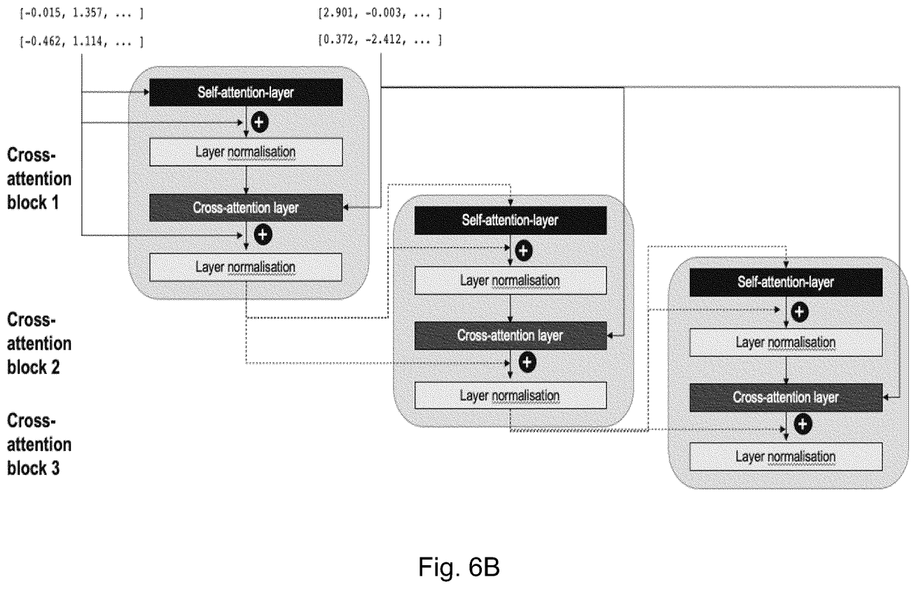

The pretrained model was then used to construct a neural network with the architecture illustrated on FIG. 6A (where AntiBERTa encoder is the encoder of the pretrained model described above), trained for a classification task as illustrated on FIG. 5B.

In the encoder module, the model separately processes an input heavy chain and an input light chain using respective copies of the pre-trained encoder. This generates a L×768 tensor for the heavy chain sequence (where L is the length of the heavy chain sequence), and a L′×768 tensor for the light chain sequence (where L′ is the length of the light chain sequence). The 768 corresponds to 768 numbers generated by the pretrained encoder (the embedding dimension). These can be understood as a set of 768 numbers that describe some contextual property of an amino acid in a heavy/light chain sequence. In practice, we process multiple heavy chains and multiple light chains at a time, for improved efficiency of processing (reduced computational time per processed pair of chains). This generates a B×max(L)×768 tensor, where B is the number of heavy chain sequences, max(L) is the maximum length of the heavy chain sequence among all heavy chain sequences in that batch. Similarly, we generate a B′×max(L′)×768 tensor for B′ number of light chains. B and B′ are often the same, and max(L) and max(L′) are not necessarily identical. These are the output of the encoder module.

The output of the encoder module is used as input to a cross-attention module. In particular, the two tensors (B×max(L)×768 and B′×max(L′)×768) are then processed through six cross-attention blocks. The first three cross-attention blocks are visualised in further detail in FIG. 6B. The subsequent blocks follow the same architecture. Each cross-attention block comprises a self-attention layer and a cross-attention layer. The self-attention and cross-attention layers each comprise of 12 attention heads. In the self-attention layer, only the heavy chain sequence embeddings are scored using the multi-head self-attention scoring mechanism described in Vaswani et al. (2017):

$$Attention ( Q , V , K ) = soft max ( \frac{{QK}^{T}}{\sqrt{{d}_{k}}} ) V$$

where the queries Q, keys K (dimensions dₖ) and values V (dimension dᵥ) are the heavy chain tensors. This is because the model was designed primarily to use features of the heavy chain to identify light chain, as the heavy chain sequence is more often available and believed to be more important in determining function. However, the roles of the light and heavy chains could be reversed. The self-attended heavy chain sequence embeddings are then layer-normalised, and form a residual connection with the unattended heavy chain input embeddings (i.e. the heavy chain input embeddings prior to application of the attention and normalisation). This is thought to compensate for the dampening of signal that may otherwise occur through layer normalisation. Layer normalisation and residual connections are described in relation to the encoder stack in Vaswani et al., 2017. Residual connections are simple sums, represented by the “+” sign in FIG. 6B. The cross-attention layer performs the same calculation as self-attention, except that the Q tensor is from the self-attended heavy chain embedding, while K and V are from the light chain. This cross-attention layer itself is similar to the attention mechanism in the decoder of the Vaswani et al. 2017 transformer method, with some key differences. In particular, the cross-attention layer does not use any masking, nor does it have a layer that generates tokens. Indeed, contrary to the model in Vaswani et al., the present model is not used to generate next position predictions. In the context in which the decoder is used in the present methods, the masking (which aims to ensure that the model does not “look forward” further than the current position to make next token predictions) and the layer that generates tokens are not useful in the present context. Following cross-attention, the output is layer-normalised, and forms a residual connection with the unattended heavy chain input again. This residual, normalised, cross-attended output is used as the “heavy chain” input for the next cross-attention block, and the process is repeated. The output is a B×max(L)×768 tensor.

The idea behind the cross-attention block is that the self-attention layer learns pairwise importance between positions within the heavy chain, while the cross-attention layer learns pairwise importance between each heavy chain position with respect to light chain positions. The output of the cross-attention module is used as input to a classifier module. In particular, after the six cross-attention blocks, the output is processed by an attention pooling mechanism. Attention pooling is described in Safari et al. (2020). Essentially, the idea is to compress a variable length tensor, B×max(L)×768, into a fixed shape, B×768. The attention pooling can be conceptualised as a weighted average. Other pooling mechanisms could be used. For example, pooling can be done by taking the embedding of the start token. However, this is thought to be less advantageous as it leads to a large loss of information. Alternatively, an unweighted average can be used. This retains more information than using only the embedding of the start token, but does not have the same ability to place different emphasis on different positions as the use of a weighted average. More complex pooling mechanisms may also be used, albeit with more burdensome training. Thus, the choice of a suitable pooling mechanism may be made to strike a balance between preserving information and requiring additional parameters to be trained, depending in part on the amount of available training data and computing power available for training. The attention-pooled tensor is then processed by a three-layer neural network, comprising of: (1) A sigmoid linear unit (also known as Swish unit, described in Ramachandran et al., 2017) that reduces the dimensionality of the input from B×768 to B×256, followed by a dropout at a rate of 0.25 (note that other dropout rates can be used, and are typically evaluated empirically; dropout rates between 0.1 and 0.3 were found to be suitable in the present context); (2) a rectified linear unit (ReLU) that further reduces the dimensionality of the Swish-activated input from B×256 to B×64, followed by a dropout rate of 0.25; (3) the ReLU-processed output is then passed to a sigmoid layer that computes a score between 0 and 1. Note that other choices of activation functions may be used instead or in addition to the combination of Swish and ReLUs, such as e.g. using only Swish layers, using only ReLU layers, using Tan H layers instead or in combination with Swich and/or ReLU layers, etc. These activation functions advantageously introduce nonlinearity via trainable parameters to provide flexibility to best fit the data. As such, the choice of activation functions can be guided at least to some extent by the expected behaviour of the data, and different possible choices are typically evaluated empirically, to identify a choice that best fits the data. The score can be viewed, in practice, as the probability of a heavy chain pairing with a light chain. In particular, given the nature in which we generate the negative dataset, a number being closer to 0 indicates that the pair is more reminiscent of a pair that is found in a randomly shuffled dataset, while a number closer to 1 indicates that the pair is more reminiscent of a pair that is found in a genuinely paired dataset from e.g. single-cell sequencing. As illustrated on FIG. 5B, the training data comprises true native pairs (with a training label of ‘1’) and random pairs (with a training label of ‘0’), and the model is trained to predict scores as close as possible to 1 for the latter and as close as possible to 0 for the former. It is also possible for the training data to comprise pairs associated with non-binary labels, such as e.g. a continuous score indicative of “nativeness” or “functionality”. Such a score may for example reflect functional information associated with known pairs, such as e.g. binding affinity, or any other metric associated with binding strength.

The full classifier model is trained using the following regime. Training was performed for a total of 10 epochs, allowing a peak learning rate of 3e-5, with a weight decay of 0.1, and a cosine learning rate schedule. For the first 5 epochs, the parameters of the encoder module, which generates the embeddings, are “frozen”. This means that in the first five epochs, only the cross-attention block, the attention pooling, and the final three-layer neural network can be optimised, and the encoder block remains unchanged. This advantageously enables better generalisation. For the remainder of the training, the parameters of the encoder module are allowed to vary (although both copies of the pre-trained encoder are varied at the same time, i.e. they remain exact copies of each other). This approach enables the training to initially focus on training the classification part of the model, only allowing tweaks to the encoding part of the model at a later stage when learning rates are lower. The training for the classification task used binary cross-entropy as a loss function.

### Model Architecture—Alternative

An alternative approach was designed which does not use the cross-attention module described above. In this approach, the pretrained encoder module has a longer maximum length than that described above, and takes as input the concatenation of a heavy and light chain sequence. Such a model embeds both the heavy and light chain jointly, as opposed to generating separate embeddings. This is then used as input to a classifier module as described above, comprising an attention pooling layer, Swish, ReLU and softmax layers. The pretraining of the encoder module could use masked language modelling in a similar way as explained above. Note that the encoder already contains an attention mechanism that can attend to the heavy and light chains so an additional cross-attention module may not be used. The fine-tuning of the full model (including the pretrained encoder an the classifier module) may be performed in a similar way as explained above, i.e. using a first period in which the parameters of the encoder module are frozen and training focusses on the classifier module, followed by a second period in which the parameters of the encoder module are also allowed to vary.

### Results

The training of a classifier model to predict whether two chains are likely to pair imposes some limitations on the amount of training data available, as paired heavy-light chain data is required. This type of data is available in relatively limited amounts, and is further limited in its content by providing V and J-gene identifiers instead of full sequences (thereby effectively limiting the prediction to the germline sequences for these sections). In order to get around these limitations, a model comprising an encoder that are is trained on much bigger datasets of unpaired heavy chain sequences and light chain sequences (42.3 million and 15.3 million sequences respectively) was built.

To evaluate the model, the inventors used the datasets from King et al., Setliff et al., and Eccles et al. While the in house test set of paired data (made of 223912 pairs) could also be used for this purpose, the inventors chose to evaluate on the three sets above as these are outside of their samples and provide a more rigorous reflection of model generalisation. Results of the in house test set are expected to be at least as good as those shown below for the independent data.

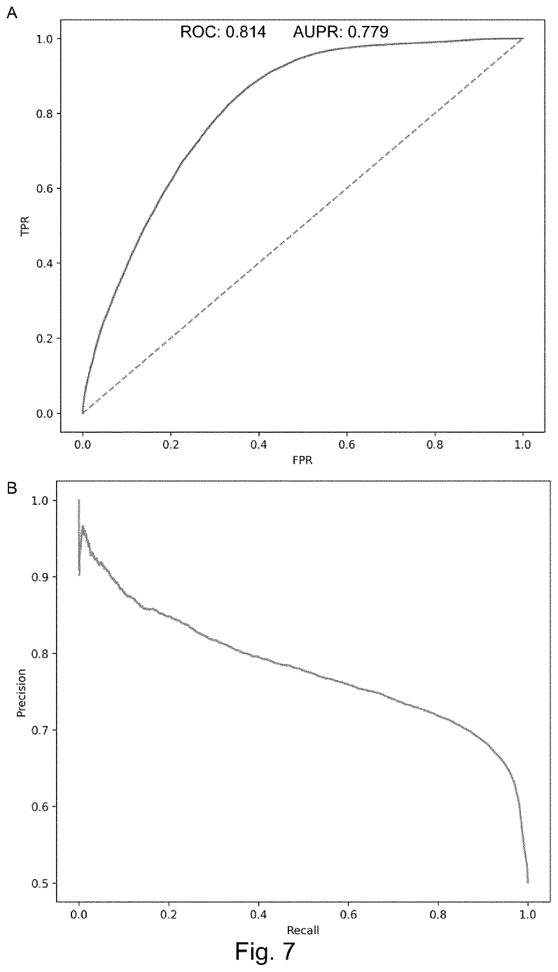

To approximate the envisioned use case of pairing a bulk heavy chain sequencing dataset with a bulk light chain sequencing dataset, more “negative” data was generated in the same way as for training the model, repeating the randomisation process multiple times for each test set. Thus, randomly paired sequences were mixed with truly paired sequences. Performance is measured by using the area under the receiver operating curve (ROC) score. A ROC score of 0.5 indicates that the model generates random predictions, while a ROC score of 1.0 indicates that the model generates perfect predictions. The ROC scores for the different sets, using the same randomisation protocol as described above, are provided in Table 1. The ROC curve for the biggest one of these datasets (King) is shown on FIG. 7A. FIG. 7B shows the corresponding Precision-Recall (PR) curve. The PR curve shows the trade-off between precision (positive predictive value, fraction of true pairs amongst the predicted pairs) and recall (sensitivity, fraction of the true pairs that were predicted as pairs), as the classification threshold is varied between 0 and 1. The area under the PR curve (AUPR) provides a useful indication of the performance of the model in finding the true pairs in an imbalanced case (i.e. where many more candidates that do not pair are expected than candidates that do truly pair). The AUPR is to be compared to the fraction of positives, which is 50% in the illustrated example. Thus, the data shows that the model performs very well (significantly better than random) at distinguishing between true pairs and random pairs. Additionally, the ROC curve can be used to identify a probability threshold that results in a desired trade-off between false positives and true positives. A trade-off that is advantageous may depend on the context such as e.g. the importance of not missing true positive pairings vs. the ability of any subsequent testing steps (whether in silico or in vitro) to accommodate larger amounts of candidate pairs. For example, a probability threshold of approximately 0.7, 0.75, 0.8, 0.85, 0.9, or 0.95 may be advantageous. In embodiments, a threshold of approximately 0.8 is used.

TABLE 1
ROC scores for test datasets (50% random pairs).

| Dataset | Dataset size | AUROC |
|---|---|---|
| Eccles | 741 positive, 741 negative | 0.802 |
| King | 30332 positive, 30332 negative | 0.814 |
| Setliff | 4944 positive, 4944 negative | 0.835 |

The inventors additionally experimented with a greater proportion of negative pairs, and the results of this are shown in Table 2.

TABLE 2
ROC scores for test datasets (70% random pairs).

| Dataset | Dataset size | AUROC |
|---|---|---|
| Eccles | 741 positive, 1770 negative | 0.805 |
| King | 30332 positive, 73759 negative | 0.813 |
| Setliff | 4944 positive, 24720 negative | 0.831 |

This data shows that the prediction performance is stable even when trying to identify true pairs amongst a greater amount of random pairs. This indicates that the model would likely perform well in a real life situation where amongst a set of candidate pairs, more are expected to be random pairs than true pairs. The data further shows stable performance across the 3 test datasets, indicating that the model shows good generalisability at least within the same species. Thus, the probabilities of being a true pair vs a random pair provided by the model can be used to compare candidate pairs, such as e.g. by ranking or otherwise prioritising candidate pairs based on this probability.

### Example 2—FAbCon for Pairing Prediction

In this example, an alternative architecture was used, which uses a generative language model. This is a decoder only model pretrained for a next token prediction task (rather than an encoder only model pretrained for a masked language modelling task as in Example 1).

FabCon is an antibody-specific large language model (LLM) based on the Falcon LLM from natural language processing (Penedo, G. et al. 2023). FAbCon is pre-trained using causal language modeling (CLM) on 779.4 million unpaired and paired antibody sequences. It is then fine-tuned for the pairing task.

### Methods

Datasets. For pre-training FAbCon models using the next-token prediction task, we collected a dataset of 823.7 million sequences (821.2 million unpaired, 2.5 million paired). This was split into 95% (777 million unpaired sequences and 2.4 million paired sequences) for pre-training the FAbCon models, and the remaining 5% (44.3 million unpaired sequences and 0.1 million paired sequences) was used for evaluating the progress of pre-training.

In more detail, the Observed Antibody Space (OAS) database was downloaded on 23^{rd} February 2023. Samples were pre-filtered in a similar procedure as previously discussed (Bachas et al. 2022) For example, B cell receptors derived from pre-B-cell samples were not used. However, we retained all sequences regardless of count. In total, we used 1.47 billion unpaired sequences (i.e., heavy chain or light chain only). In addition, we supplemented this corpus with a set of proprietary B cell receptor sequences from 376 libraries, bringing the total number of sequences to 1.54 billion. We then used Linclust (Steinegger, M. & Söding, J) to cluster the dataset at 90% sequence identity across the VH or VL domains. After clustering, 777.8 million sequences were retained from OAS, while 43.4 million sequences were retained from our proprietary data. In addition to the unpaired data, we also use heavy-light chain paired sequences for pre-training. We first combined 1.5 million publicly available paired sequences (Jaffe et al. 2022), and an in-house dataset of 1.4 million paired sequences. Due to the paucity of paired data, we applied a 99% redundancy criterion for paired data. This led to a new total of 2.5 million sequences, of which 1.4 million are from the Jaffe et al. dataset, and 1.1 million are from our in-house data. The final dataset comprising of 823.7 million sequences (821.2 million unpaired, 2.5 million paired) was split into 95% (777 million unpaired sequences and 2.4 million paired sequences) and 5% (44.3 million unpaired sequences and 0.1 million paired sequences) for training and evaluating CLM, respectively.

1791293 sequences were used for fine-tuning: this comprises 895765 sequences from single B cell sequencing, which we consider as ‘true’ pairs. An additional 895528 sequences were generated by random matching of heavy and light chains in the single B cell sequencing data—we consider these as ‘false’ pairs. Following training, the fine-tuned model was evaluated on three separate, external single B cell sequencing datasets.

Tokenisation. Sequences are tokenised at the amino acid level, where each amino acid is a token. Antibody heavy chains are preceded by the [symbol] token, while light chains are prefixed by the [symbol] token. FAbCon can accept both unpaired and paired chains as input. In the case of paired chains, the input is effectively a longer, single amino acid sequence that includes both [symbol] and [symbol] tokens (e.g. [symbol] QVQ . . . TVSS [symbol] DIV . . . VEIK). FAbCon accepts 26 tokens: the 20 amino acids, [symbol] , [symbol] , <|endoftext|>, <|unk|>, <|pad|>, and <|mask|>.

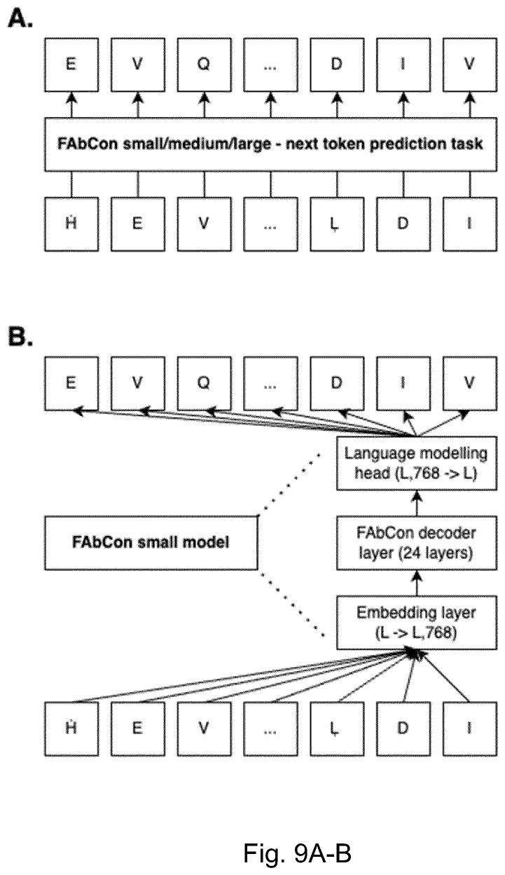

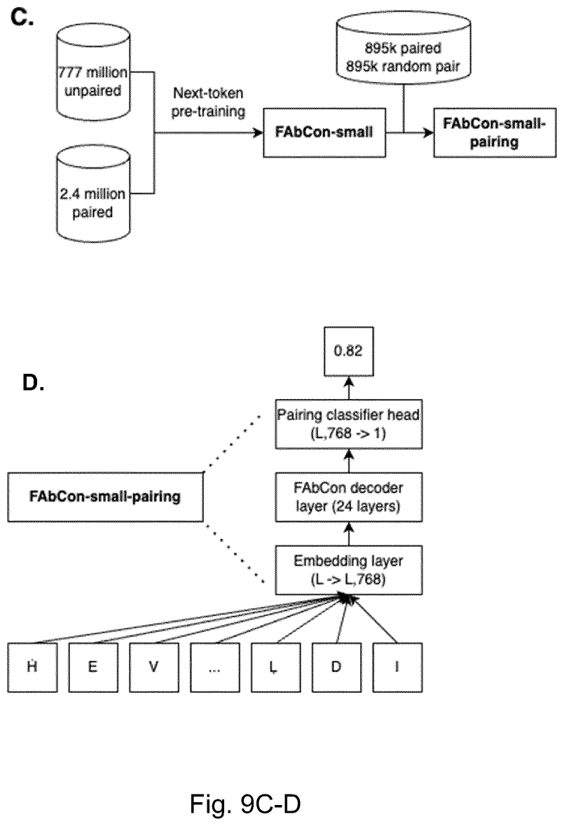

Pre-training. FAbCon is a generative transformer model based on the Falcon architecture (Penedo et al. 2023), and available at falconllm.tii.ae/falcon-models.html https://huggingface.co/docs/transformers/main/model_doc/falcon. This is a decoder-only autoregressive transformer model with an architecture based on GPT-3 (Brown et al. 2020), with ALiBi positional encodings (Press et al. 2021) and FlashAttention (Dao et al., 2022). The Falcon models use multi-query attention, which shares keys, and value embeddings across attention heads, reducing memory costs for pretraining and inference. We have pre-trained three FAbCon variants on the next-token prediction task (FIG. 9A): a 144 million parameter variant, a 300 million parameter, and a 2.4 billion parameter variant. All FAbCon variants were pre-trained using 48 NVIDIA A100's (80 GB) from NVIDIA's DGX SuperCloud (Cambridge-1 environment). The configuration for each variant is listed in Table 3. For next-token prediction, the model is tasked with predicting the amino acid sequence in a left-to-right manner. Only the preceding residues in the N-terminus are used for informing the prediction of subsequent amino acids. Each FAbCon model has a maximum context window of 256 (i.e. maximum sequence length of 256). During pre-training, both unpaired and paired antibody sequences can form one mini-batch.

FAbCon variants were pre-trained using the CLM objective, where the model is tasked with autoregressively predicting each residue by attending only to the previous residues in the sequence. More formally, the model was tasked with predicting the probability of the amino acid residue at position t, Tt:

P(rₜ|r₁,r₂, . . . ,rₜ₋₁).

During pre-training, the model parameters were updated via gradient-based optimisation in order to minimize the cross-entropy loss as given by

$${L}_{CE} = - \frac{1}{N} {\sum }_{t = 1}^{T} {\sum }_{i = 1}^{V} {L}_{t , i} log {P ( {r}_{t} \vert {r}_{1} , {r}_{2} , ... , {r}_{t - 1} )}_{i}$$

where N is the batch size, T is the sequence length, V is the vocabulary size, and L_{t,i} is the label for the residue at position t, and the ith word in the vocabulary (here comprising 26 words for the 20 amino acids, <|endoftext|>, <|unk|>, <|pad|>, <|mask|>, [symbol] and [symbol] prefixes).

As mentioned above all three FAbCon variants feature flash attention and multi-query attention to increase pre-training speed and reduce memory footprint. For each variant, we adopted a “deep narrow” architecture where we increased the number of layers and maintained a relatively lower embedding dimensionality. Optimisation used the Fused AdamW optimiser with a peak learning rate was 2e-5, with a cosine learning rate schedule, 200 k steps, a weight decay of 0.01, gradient norm clipping of 1.0.

TABLE 3
Configuration of FAbCon models.

| | FAbCon- | FAbCon- | FAbCon- |
|---|---|---|---|
| | small | medium | large |
| Number of layers | 24 | 28 | 56 |
| Number of attention heads | 12 | 16 | 32 |
| Embedding dimension | 768 | 1024 | 2048 |
| Feed-forward dimension | 3072 | 4096 | 8192 |
| Total learnable parameters | 144 million | 300 million | 2.4 billion |

Fine-tuning. FIGS. 9B and 9D shows the different architectures for FAbCon for pre-training (FIG. 9B) against the classifier model that discriminates whether an input sequence is a genuine or false pairing (FIG. 9D). The difference between FAbCon-small (a generative language model) and FAbCon-small-pairing (pairing classifier model) is the final “head” layer at the end. FAbCon-small-pairing uses the representations from the decoder layers to output a probability that an input sequence forms a pair. This architecture can be applied in a similar manner for FAbCon-medium and FAbCon-large; for practical reasons, we only show results for fine-tuning the FAbCon-small model.

FAbCon-small was fine-tuned for 10 epochs, with a peak learning rate of 5e-5 after a 5% warmup. Dropout was set at 0.1, and weight decay at 0.01. We used the checkpoint of the model with the lowest validation set loss, which corresponded to the 5th epoch. No model freezing was implemented here. FIG. 9C provide the overall view of how the model was pre-trained, then fine-tuned for the purpose of pairing.

### Results

The results of the process described above are shown in Tables 4 and 5 below. These show very good prediction accuracy albeit slightly lower than in Example 1 (although similar performance is expected to be achievable with further optimisation).

TABLE 4
ROC scores for test datasets (50% random pairs).

| Dataset | Dataset size | AUROC |
|---|---|---|
| Eccles | 741 positive, 741 negative | 0.790 |
| King | 30332 positive, 30332 negative | 0.803 |
| Setliff | 4944 positive, 4944 negative | 0.828 |

TABLE 4
ROC scores for test datasets (70% random pairs).

| Dataset | Dataset size | AUROC |
|---|---|---|
| Eccles | 741 positive, 1770 negative | 0.797 |
| King | 30332 positive, 73759 negative | 0.802 |
| Setliff | 4944 positive, 24720 negative | 0.825 |

The same model was also fine-tuned for antibody-antigen binding prediction (classification of pairs of sequences as binder or not binder for a specific antigen; data not shown) where it showed exceptionally strong performance (mean average precision between 0.851 across 3 different antigens tested, increasing to 0.851 and 0.883 for the medium and large models). This shows that this model is good at discriminating binders from non-binders, further demonstrating that the encoding module of models as described herein can learn information that discriminates functional pairs from non-functional pairs.

### Example 3—Discussion

This example describes a machine-learning, NLP-inspired approach to the problem of BCR heavy-light chain pairing. The deep-learning based approach described herein provides the benefit of covering the BCR repertoire as deeply as possible, while also eliminating the need for bulk light chain sequencing or single cell sequencing. Further, the approach is capable of learning general features of pairings from a set of training data and to use this learning to predict pairings for previously unseen chains. This is likely to be advantageous in many cases considering the extreme diversity of the BCR repertoire, but particularly so in the context of applications such as identifying specific antibodies that may underlie a desired phenotype in an individual, or other rare antibodies.

The approach makes the most of available training data through a combination of using larger training data sets of unpaired full-length sequences for pretraining of a language model, and smaller paired data sets with potentially less extensive sequence coverage for fine-tuning of a final model comprising the encoding module (this is the part of the language model that provides the latent representation of the input sequences, e.g. encoder or decoder, depending on model architecture) and a classifier module.

Additionally, the approach provides a way to evaluate candidate chains that are “genuine” chains, thereby increasing the changes of functional pairs being identified. For example, the approach would be ideally suited to situations where the repertoires of both pairs were identified (or at least a subset of the repertoire) but the pairing information between the two repertoires was not available. Indeed, in such cases the present approach is able to take advantage of the knowledge that the native partner of a candidate chain is likely to be present in a set of candidates. By contrast, an approach that generates a chain for pairing “de novo” may generate a prediction that would not fold appropriately for a chain of this type, or that would not form a functional pair with any chain. Note that approaches that generate de novo predictions may be used in combination with approaches of the present invention, for example by using the former to provide candidates for evaluation by the latter.

In order to apply bulk heavy chain repertoire analyses to antibody discovery, the issue of light chain pairing remains pertinent. The deep learning-based approach described herein enables the identification of light chains that are likely to pair with any heavy chain of interest, by evaluating known or otherwise generated candidate light chains for pairing, with high in silico accuracy. This approach thus has the potential to fill gaps in light chain pairing information, thus enabling therapeutic antibody discovery and a better understanding of the immune system.

Finally, while the approach was described in the context of identifying a light chain pairing for a heavy chain query, it is also applicable to the reverse problem of identifying a heavy chain pairing for a light chain query, as well as to the pairing of other immune molecule dimers. This is a less frequent problem as antibodies are widespread therapeutic, diagnostic and research tools, and in the particular context of antibodies, heavy chain sequencing is more common and the heavy chain is believed to play a more important part in determining specificity and affinity.

### REFERENCES

Vander Heiden et al., 2017. Dysregulation of B Cell Repertoire Formation in Myasthenia Gravis Patients Revealed through Deep Sequencing. J Immunol. 2017 Feb. 15; 198 (4): 1460-1473.
Bashford-Rogers et al, 2019. Analysis of the B cell receptor repertoire in six immune-mediated diseases. Nature volume 574, pages 122-126 (2019).
Nielsen et al., 2020. Human B Cell Clonal Expansion and Convergent Antibody Responses to SARS-COV-2. bioRxiv. Preprint. 2020 Jul. 9. doi: 10.1101/2020.07.08.194456.
Simonich et al., 2019. Kappa chain maturation helps drive rapid development of an infant HIV-1 broadly neutralizing antibody lineage. Nature Communications volume 10, Article number: 2190 (2019).
Krawczyk et al., 2019. Looking for therapeutic antibodies in next-generation sequencing repositories. mAbs. Volume 11, 2019-Issue 7, Pages 1197-1205.
Galson et al., 2020. Deep Sequencing of B Cell Receptor Repertoires From COVID-19 Patients Reveals Strong Convergent Immune Signatures. Front. Immunol., 15 Dec. 2020. doi.org/10.3389/fimmu.2020.605170.
Mora and Walczak, 2019. How many different clonotypes do immune repertoires contain? Current Opinion in Systems Biology. Volume 18, December 2019, Pages 104-110
Kovaltsuk et al., 2018. Observed Antibody Space: A Resource for Data Mining Next-Generation Sequencing of Antibody Repertoires. J Immunol Oct. 15, 2018, 201 (8) 2502-2509.
Teplyakov et al., 2016. Structural diversity in a human antibody germline library. MAbs. August-September 2016; 8 (6): 1045-63.
Glanville et al., 2009. Precise determination of the diversity of a combinatorial antibody library gives insight into the human immunoglobulin repertoire. PNAS Dec. 1, 2009 106 (48) 20216-20221.
Jayaram et al., 2012. Germline V H/V L pairing in antibodies. Protein Engineering, Design and Selection, Volume 25, Issue 10, October 2012, Pages 523-530.
Ling et al., 2018. Effect of VH-VL Families in Pertuzumab and Trastuzumab Recombinant Production, Her2 and FcγIIA Binding. Front. Immunol., 12 Mar. 2018. doi.org/10.3389/fimmu.2018.00469
DeKosky et al., 2016. Large-scale sequence and structural comparisons of human naive and antigen-experienced antibody repertoires. PNAS May 10, 2016 113 (19) E2636-E2645.
King et al., 2021. Single-cell analysis of human B cell maturation predicts how antibody class switching shapes selection dynamics. Science Immunology 12 Feb. 2021. Vol. 6, Issue 56, eabe6291
Eccles et al., 2020. T-bet+ Memory B Cells Link to Local Cross-Reactive IgG upon Human Rhinovirus Infection. Cell Reports Volume 30, Issue 2, 14 Jan. 2020, Pages 351-366.e7
Setliff et al., 2019.High-Throughput Mapping of B Cell Receptor Sequences to Antigen Specificity. Cell Volume 179, Issue 7, 12 Dec. 2019, Pages 1636-1646.e15
Reddy et al., 2010. Monoclonal antibodies isolated without screening by analyzing the variable-gene repertoire of plasma cells. Nature Biotechnology volume 28, pages 965-969 (2010).
Zhu et al., 2013. Mining the antibodyome for HIV-1-neutralizing antibodies with next-generation sequencing and phylogenetic pairing of heavy/light chains. PNAS. 2013 Apr. 16; 110 (16): 6470-5.
Raybould et al., 2021. Public Baseline and shared response structures support the theory of antibody repertoire functional commonality. PLOS Comput Biol 17 (3): e1008781.
Rakocevic et al., 2021. The landscape of high-affinity human antibodies against intratumoral antigens. bioRxiv. 8 Feb. 2021. doi.org/10.1101/2021.02.06.430058
Vaswani et al., 2017. Attention Is All You Need. arXiv: 1706.03762
Devlin, Jacob, et al. “Bert: Pre-training of deep bidirectional transformers for language understanding.” arXiv preprint arXiv: 1810.04805 (2018).
Radford et al., 2019. Language Models are Unsupervised Multitask Learners. https://openai.com/blog/better-language-models/
Liu et al., 2019. ROBERTa: A Robustly Optimized BERT Pretraining Approach. arXiv: 1907.11692
Rothe et al., 2020. Leveraging Pre-trained Checkpoints for Sequence Generation Tasks. arXiv: 1907.12461
Dunbar and Deane, 2016. ANARCI: antigen receptor numbering and receptor classification. Bioinformatics. 2016 Jan. 15; 32 (2): 298-300.
Rees, 2020. Understanding the human antibody repertoire. MAbs. January-December 2020; 12 (1): 1729683.
Ye et al., 2013. IgBLAST: an immunoglobulin variable domain sequence analysis tool. Nucleic Acids Res. 2013 July; 41 (Web Server issue): W34-40.
Carter J A et al. Single T Cell Sequencing Demonstrates the Functional Role of αβ TCR Pairing in Cell Lineage and Antigen Specificity. Frontiers in Immunology. Vol. 10. 2019, p. 1516.
Zheng G X Y, Terry J M, Belgrader P, Ryvkin P, Bent Z W, Wilson R, et al. Massively parallel digital transcriptional profiling of single cells. Nat Commun. (2017) 8:14049.
Howie B, Sherwood A M, Berkebile A D, Berka J, Emerson R O, Williamson D W, et al. High-throughput pairing of T cell receptor α and β sequences. Sci Transl Med. (2015) 7: 301ra131.
Eve Richardson, Jacob D. Galson, Paul Kellam, Dominic F. Kelly, Sarah E. Smith, Anne Palser, Simon Watson & Charlotte M. Deane (2021) A computational method for immune repertoire mining that identifies novel binders from different clonotypes, demonstrated by identifying anti-pertussis toxoid antibodies, mAbs, 13:1.
Yi-Chun Hsiao, Yonglei Shang, Danielle M. DiCara, Angie Yee, Joyce Lai, Si Hyun Kim, Diego Ellerman, Racquel Corpuz, Yongmei Chen, Sharmila Rajan, Hao Cai, Yan Wu, Dhaya Seshasayee & Isidro Hotzel (2019) Immune repertoire mining for rapid affinity optimization of mouse monoclonal antibodies, mAbs, 11:4, 735-746.
Warszawski S, Borenstein Katz A, Lipsh R, Khmelnitsky L, Ben Nissan G, Javitt G, et al. (2019) Optimizing antibody affinity and stability by the automated design of the variable light-heavy chain interfaces. PLOS Comput Biol 15 (8): e1007207.
Seeliger D, Schulz P, Litzenburger T, Spitz J, Hoerer S, Blech M, Enenkel B, Studts J M, Garidel P, Karow A R. Boosting antibody developability through rational sequence optimization. MAbs. 2015; 7 (3): 505-15. doi: 10.1080/19420862.2015.1017695.
Mason, D. M., Friedensohn, S., Weber, C. R. et al. Optimization of therapeutic antibodies by predicting antigen specificity from antibody sequence via deep learning. Nat Biomed Eng (2021).
Leem, J., Mitchell, L. S., Farmery, James H. R., Barton, J., Galson, J. D. Deciphering the language of antibodies using self-supervised learning. Patterns 3, 100513. Jul. 8, 2022.
Safari, Pooyan, Miquel India, and Javier Hernando. “Self-attention encoding and pooling for speaker recognition.” arXiv preprint arXiv: 2008.01077 (2020).
Ramachandran, Prajit, Barret Zoph, and Quoc V. Le. “Searching for activation functions.” arXiv preprint arXiv: 1710.05941 (2017).
Shaw, Peter, Jakob Uszkoreit, and Ashish Vaswani. “Self-attention with relative position representations.” arXiv preprint arXiv: 1803.02155 (2018). Su, Jianlin, et al. “Roformer: Enhanced transformer rotary position embedding.” arXiv preprint with arXiv: 2104.09864 (2021).
Nijkamp, Erik, et al. “ProGen2: exploring the boundaries of protein language models.” arXiv preprint arXiv: 2206.13517 (2022).
Cédric R Weber, Rahmad Akbar, Alexander Yermanos, Milena Pavlović, Igor Snapkov, Geir K Sandve, Sai T Reddy, Victor Greiff, immuneSIM: tunable multi-feature simulation of B- and T-cell receptor repertoires for immunoinformatics benchmarking, Bioinformatics, Volume 36, Issue 11, June 2020, Pages 3594-3596.
Penedo, G. et al. The RefinedWeb Dataset for Falcon LLM: Outperforming Curated Corpora with Web Data, and Web Data Only. arXiv (2023) doi: 10.48550/arxiv.2306.01116.
Steinegger, M. & Söding, J. Clustering huge protein sequence sets in linear time. Nat. Commun. 9, 2542 (2018).
Jaffe, D. B. et al. Functional antibodies exhibit light chain coherence. Nature 611, 352-357 (2022).
Brown, T., Mann, B., Ryder, N., Subbiah, M., Kaplan, J. D., Dhariwal, P., Neelakantan, A., Shyam, P., Sastry, G., Askell, A., et al. Language models are few-shot learners. Advances in neural information processing systems, 33:1877-1901, 2020.
Dao, T., Fu, D. Y., Ermon, S., Rudra, A., and Re, C. Flashattention: Fast and memory-efficient exact attention with io-awareness. In Advances in Neural Information Processing Systems, 2022.
Press, O., Smith, N., and Lewis, M. Train short, test long: Attention with linear biases enables input length extrapolation. In International Conference on Learning Representations, 2021.
Touvron H. et al. 2023. Llama 2: Open Foundation and Fine-Tuned Chat Models arXiv: 2307.09288v2. 19 Jul. 2023
Mandar Joshi, Danqi Chen, Yinhan Liu, Daniel S. Weld, Luke Zettlemoyer, Omer Levy. SpanBERT: Improving Pre-training by Representing and Predicting Spans. arXiv: 1907.10529v3. 18 Jan. 2020

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Various modifications and variations of the described compositions, methods, and uses of the technology will be apparent to those skilled in the art without departing from the scope and spirit of the technology as described. Any sub-titles herein are included for convenience only and are not to be construed as limiting the disclosure in any way. Other aspects and embodiments of the invention provide the aspects and embodiments described above with the term “comprising” replaced by the term “consisting of” or “consisting essentially of”, unless the context dictates otherwise.

The methods of any embodiments described herein may be provided as computer programs or as computer program products or computer readable media carrying a computer program which is arranged, when run on a computer, to perform the method(s) described above.

Unless context dictates otherwise, the descriptions and definitions of the features set out above are not limited to any particular aspect or embodiment of the invention and apply equally to all aspects and embodiments which are described.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps. “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

1. A computer-implemented method for determining whether a pair of protein chains comprising a first chain and a second chain are likely to form a functional antigen-binding protein, the method comprising:
providing a query pair of sequences comprising the sequence of the first protein chain and the sequence of the second protein chain as input to a deep learning model configured to take as input a pair of protein chain sequences and to produce as output a score indicative of the probability that the pair of protein chains will form a functional antigen-binding protein, wherein the deep learning model comprises an encoder module and a classifier module, and wherein the deep learning module has been trained using paired training sequences from known antigen-binding proteins.

2. A computer-implemented method of identifying an antigen-binding protein comprising a first protein chain and a second protein chain, the method comprising:
providing a query first protein chain, and
identifying a second protein chain by:
providing one or more candidate second protein chain sequences; and
determining whether the one or more candidate second chain sequences are likely to form a functional antigen-binding protein with the query first protein chain by: providing as input to a deep learning model one or more query pair of sequences each comprising (i) the sequence of the first protein chain and (ii) a candidate second protein chain sequence, wherein the deep learning model is configured to take as input one or more pairs of protein chain sequences and to produce as output a score indicative of the probability that each pair of protein chains will form a functional antigen-binding protein or information derived therefrom, wherein the deep learning model comprises an encoder module and a classifier module, and wherein the deep learning module has been trained using paired training sequences from known antigen-binding proteins, optionally wherein identifying a second protein chain comprises providing a plurality of candidate second chain sequences and the information derived from the score comprises a ranking of the pairs of protein chain sequences wherein pairs of protein sequences that are more likely to form a functional antigen-binding protein are ranked higher than pairs of protein sequences that are less likely to form a functional antigen-binding protein.

3. The method of claim 1 of claim 2, wherein the antigen-binding protein comprises:
(i) a heavy-light chain pair, wherein the first chain is a heavy chain or a light chain, and the second chain is a light chain or a heavy chain, optionally wherein the first chain is a heavy chain and the second chain is a light chain; or
(ii) an αβ chain pair, wherein the first chain is a β chain or an α chain, and the second chain is an α chain or a β chain, optionally wherein the first chain is a β chain and the second chain is an α chain; or
(ii) a γδ chain pair, wherein the first chain is a δ chain or a γ chain, and the second chain is a γ chain or a δ chain, optionally wherein the first chain is a δ chain and the second chain is a γ chain.

4. The method of any preceding claim, wherein the encoder module comprises one or two encoders that have been pretrained using training sequences from unpaired protein chains from known antigen-binding proteins, or a decoder that has been pretrained using training sequences comprising paired and unpaired protein chains from known antigen-binding proteins.

5. The method of any preceding claim, wherein the encoder module comprises:
one or two encoders of a sequence-to-sequence model or a decoder of a generative language model, optionally wherein the sequence-to-sequence model or generative language model is a transformer-based model, or
one or two transformer-based encoder or decoder models.

6. The method of any preceding claim, wherein the encoder module comprises one or two encoder models of a sequence-to-sequence model, wherein the encoder models have been trained using masked language modelling, optionally wherein training the encoder models comprises training the model to replace randomly masked positions of training amino acid sequences and/or wherein 15% of positions are masked during training and/or wherein masked positions are replaced with mask tokens, a random amino acid, or the original amino acid at the position, or wherein the encoder module comprises a decoder model of a generative language model, wherein the decoder model has been trained using causal language modelling.

7. The method of any preceding claim, wherein the encoder module comprises one or two encoders that have been pretrained using training sequences comprising unpaired first and second protein chains from known antigen-binding proteins, optionally wherein the training sequences comprise at least 1 million, at least 2 million, at least 5 million, at least 10 million, at least 20 million or at least 50 million individual sequences, or wherein the encoder module comprises a decoder that has been pretrained using training sequences comprising unpaired first and second protein chains from known antigen-binding proteins and paired first and second protein chains from known antigen-binding proteins, optionally wherein the training sequences comprise at least 500 million, 600 million or 700 million individual sequences and/or at least 1, 1.5 or 2 million paired sequences.

8. The method of any preceding claim, wherein the encoder module comprises two copies of an encoder that has been pretrained using training sequences comprising unpaired first and second protein chains from known antigen-binding proteins.

9. The method of any of claims 1 to 7, wherein the encoder module comprises an encoder that has been pretrained using training sequences comprising the concatenation of a sequence from a first chain and a sequence from a second protein chain from known antigen-binding proteins, wherein the concatenated sequences comprise sequences from unpaired protein chains, or wherein the encoder module comprises a decoder that has been pretrained using training sequences each comprising a single chain or the concatenation of a sequence from a first chain and a sequence from a second protein chain from a known antigen-binding protein.

10. The method of any of claims 1 to 8, wherein the deep learning model further comprises a cross-attention module that takes as input the output of the encoder module, and produces an output used by the classifier module to provide the core indicative of the probability that the pair of protein chains will form a functional antigen-binding protein.

11. The method of claim 10, wherein the cross-attention module comprises one or more cross-attention blocks, wherein each cross-attention block comprises a self-attention layer and a cross attention layer, and/or wherein the cross-attention module comprises a plurality of cross-attention blocks.

12. The method of claim 11, wherein the cross-attention module comprises one or more cross-attention blocks comprising a cross-attention layer and a self-attention layer, and each cross-attention layer and self-attention layer comprises a plurality of attention heads.

13. The method of any of claims 10 to 12, wherein each self-attention layer comprises one or more attention heads that attend to the output of a first encoder taking as input the first chain sequence, and/or wherein each cross-attention block further comprises a residual connection between the output of a first encoder and the output of the self-attention layer.

14. The method of any of claims 10 to 13, wherein each cross-attention layer comprises one or more attention heads that attend to: (i) the output of the self-attention layer or the output of a first encoder taking as input the first chain sequence, and (ii) the output of a second encoder taking as input the second chain sequence, and/or wherein each cross-attention block further comprises a residual connection between the output of a first encoder and the output of the cross-attention layer.

15. The method of any preceding claim, wherein the classification module comprises a softmax layer that produces a score between 0 and 1 that can be interpreted as the probability that a pair of input protein chains will form a functional antigen-binding protein, and/or wherein the classification module comprises one or more of: a dimensionality reduction layer, a regularisation mechanism, and a layer with an activation function, optionally wherein a dimensionality reduction layer comprises an attention pooling layer and/or wherein a regularisation layer comprises a dropout mechanism, and/or wherein an activation function is independently selected from Tanh, Leaky ReLU, SmeLU, GeLU Swish and ReLU.

16. The method of any preceding claim, wherein the deep learning model takes as input a plurality of query pairs of chain sequences and produces as output a respective score indicative of the probability that each query pair of protein chains will form a functional antigen-binding protein, and/or a ranking of the plurality of query pairs of chain sequences such that query pairs of protein sequences that are more likely to form a functional antigen-binding protein are ranked higher than query pairs of protein sequences that are less likely to form a functional antigen-binding protein.

17. The method of any preceding claim, wherein the paired training sequences from known antigen-binding proteins comprise paired training heavy and light chain sequences from single B cell sequencing data, and/or wherein the training data further comprises a negative set comprising randomly paired first and second protein chain sequences,
optionally wherein the randomly paired first and second protein chain sequences are obtaining by re-pairing the paired training sequences from known antigen-binding proteins and/or by randomly pairing unpaired training sequences and/or wherein the negative set comprises paired first and second protein chain sequences from antigen-binding proteins previously determined to be non-functional and/or wherein the paired training sequences are associated with a first label and the randomly paired training sequences are associated with a second label.

18. The method of any preceding claim, wherein providing a pair of protein chain sequences as input to the deep learning model comprise encoding each of the protein chain sequences using a predetermined encoding scheme, optionally wherein each amino acid is individually encoded or wherein sequences are encoded using tokens that each correspond to an individual k-mer, and/or wherein each protein chain sequence is preceded or followed by a special token indicating whether the protein chain sequence is a first or second protein chain.

19. The method of any preceding claim, wherein providing a query first protein chain or a query first protein chain of a query pair comprises:
obtaining the sequence of the query first protein chain from a user through a user interface, from a computing device, from a sequence acquisition means or a computing device associated with a sequence acquisition means, from a database or other computer readable medium; and/or
sequencing a sample comprising genetic material encoding for an antigen-binding molecule comprising the query sequence, optionally wherein obtaining the query sequence comprises performing B cell bulk sequencing of a sample comprising B cells, T cell bulk sequencing of a sample comprising T cells, or bulk sequencing of a sample comprising any other cells expressing an antigen-binding molecule comprising the query sequence, or genetic material derived therefrom, such as a B cell receptor library or a T cell receptor library; and/or
obtaining a sample comprising B cells, T cells or other cells expressing an antigen-binding molecule comprising the query sequence, or genetic material derived therefrom, such as a B cell receptor library or T cell receptor library; and/or
wherein providing one or more candidate second protein chain sequences comprises obtaining the sequences of the candidate second protein chains from a user through a user interface, from a computing device, from a sequence acquisition means or a computing device associated with a sequence acquisition means, from a database or other computer readable medium; and/or wherein the one or more candidate second protein chain sequences are known second chain protein sequences or simulated second chain protein sequences.

20. The method of any preceding claim, further comprising providing one or more identified second protein chains, a part thereof or information derived therefrom, and/or one or more scores indicative of the probability that one or more query pairs form a functional antigen-binding protein or information derived therefrom to a user through a user interface; and/or further comprising predicting a score indicative of the probability that each one or more query pairs comprising respective candidate second protein chain sequences will form a functional antigen-binding protein and identifying a candidate second protein chain sequence by applying one or more criteria on the score, optionally wherein the one or more criteria are selected from: the score being above a predetermined cutoff, the score being the highest predicted score of a set of candidate second protein chain sequences, and the score being in a predetermined top percentile of the predicted probability of a set of candidate second protein chain sequences; and/or
wherein the core is a probability that the pair of protein chains will form a functional antigen-binding protein.

21. A method of providing antigen-binding protein chain pairings for a plurality of query sequences comprising a first chain sequence, the method comprising: performing the method of any of claims 2 to 20 for each of the query sequences, optionally wherein the plurality of query sequences are heavy or light chain sequences obtained by bulk B cell repertoire sequencing.

22. A method of providing an antigen-binding protein having a desired property, the method comprising:
providing one or more query sequences comprising a first chain sequence, wherein at least one of the one or more query sequences is likely to have the desired property, and
identifying a second chain sequence for each of the one or more query sequences using the method of any of claims 2 to 20.

23. A method of providing a tool for predicting whether a pair of protein chains is likely to form a function antigen-binding protein, the method comprising:
providing training data comprising training first and second protein chain sequences from known antigen-binding proteins, and
training a deep learning model to take as input a pair of protein chain sequences and to produce as output a score indicative of the probability that the pair of protein chains will form a functional antigen-binding protein, wherein the deep learning model comprises an encoder module and a classifier module.

24. A system comprising:
a processor; and
a computer readable medium comprising instructions that, when executed by the processor, cause the processor to perform the steps of the method of any of claims 1 to 23.

25. One or more computer readable media comprising instructions that, when executed by one or more processors, cause the one or more processors to perform the steps of the method of any of claims 1 to 23.
